(19) United States
(12) Patent Application Publication
(10) Pub. No.: US 2008/0200441 A1
(43) Pub. Date: Aug. 21, 2008

(54) ESTROGEN RECEPTOR MODULATORS ASSOCIATED PHARMACEUTICAL COMPOSITIONS AND METHODS OF USE

(75) Inventors: Roberta Diaz Brinton, Rancho Palos Verdes, CA (US); Liqin Zhao, Los Angeles, CA (US)

Correspondence Address: PATREA L. PABST;PABST PATENT GROUP LLP, 400 COLONY SQUARE, SUITE 1200, 1201 PEACHTREE STREET, ATLANTA, GA, 30361, US

(73) Assignee: University of Southern California

(21) Appl. No.: 12/031,538

(22) Filed: Feb. 14, 2008

Related U.S. Application Data
(60) Provisional application No. 60/889,920, filed on Feb. 14, 2007.
(60) Provisional application No. 60/943,190, filed on Jun. 11, 2007.
(60) Provisional application No. 60/988,273, filed on Nov. 15, 2007.

(51) Int. Cl. A61K 31/56; A61P 25/28; C07D 311/02; A61K 31/351; A61K 31/135; C07C 211/00; A61K 31/122

(52) U.S. Cl. 514/182; 552/540; 549/398; 514/456; 514/646; 564/305; 568/327; 514/681

(57) ABSTRACT

Selective estrogen receptor modulators, as well their related pharmaceutical compositions and methods of use, are provided herein. These estrogen receptor modulators include compounds that primarily exhibit estrogen receptor antagonist activity or primarily exhibit selective estrogen receptor antagonist and agonist activity, i.e., SERM activity, in specific tissue types. Particular embodiments provide compounds that behave as NeuroSERMs promoting neurotrophism and neuroprotection in brain tissue. These NeuroSERMs represent a subset of the modulators compounds provided herein that can cross the blood-brain-barrier and exert estrogen receptor agonist-like effects in the brain. The compounds should be useful for treating a variety of diseases, particularly estrogen receptor-mediated diseases and disorders, such as osteoporosis, breast and endometrial cancers, atherosclerosis and Alzheimer's disease.

### CROSS REFERENCE TO RELATED APPLICATIONS

This application claims priority to U.S. Ser. No. 60/889,920, filed on Feb. 14, 2007, U.S. Ser. No. 60/943,190, filed on Jun. 11, 2007, and U.S. Ser. No. 60/988,273 filed on Nov. 15, 2007.

### FIELD OF THE INVENTION

The present disclosure generally relates to the field of selective estrogen receptor modulators, and methods of making and using thereof.

### BACKGROUND

While estrogen replacement therapy (ERT), both unopposed estrogen and estrogen/progestin in combination, has long been used in postmenopausal women to delay or reverse some of the problems associated with menopause, epidemiologic and clinical studies have uncovered potential long-term risks related to this therapy. The recently revealed risks associated with ERT have greatly increased interest in the development of estrogen alternatives that promote the beneficial effects of estrogen in brain, bone and the cardiovascular system, while not eliciting deleterious effects in other organs, particularly in breast and uterine tissue.

Such selective estrogen receptor modulators, exemplified by Tamoxifen, were first defined as estrogen receptor (ER) antagonists and used for the treatment of ER positive breast cancer. The discovery of the positive effect of Tamoxifen in bone associated with an ER agonizing action accelerated the development of second and third generation selective estrogen receptor modulators. This new class of molecules was accordingly redefined as selective ER modulators, or SERMs, which differentially bind to and modulate ER in a tissue-specific manner. The biochemical basis for the tissue specificity of the action of SERMs still remains unresolved, but increasing evidence suggests that the ER agonist or antagonist action of an individual SERM in distinct tissues depends on several factors including (1) differential expression of ER subtypes or isoforms; (2) tissue co-activators and co-repressors (coregulators); (3) the ER conformation following SERM binding, which is responsible for the variable recruitment of coregulators needed for ER-mediated gene transcription; and (4) the variable activation of cellular second messenger pathways leading to indirect genomic effects or ER-independent actions.

Of particular interest is the discovery and development of SERMs that behave as agonists in brain tissue and promote neurological function. The discovery and development of ideal and effective SERMs that would exert estrogen agonist activity in the brain, while exerting estrogen antagonist activity in breast and uterus, would be of great interest in oncology and medicine. This ideal SERM would have tremendous therapeutic value in treating breast and uterine cancers, while promoting neurological function in a population at risk for losing neurological capacity and memory function, i.e., postmenopausal women.

ICI 182,780 has been shown to have estrogen receptor agonist-like effects in hippocampal neurons of the brain. ICI 182,780 (Faslodex) is a derivative of 17β estradiol with a long hydrophobic side chain at the 7-α position. The structure of IC 182,780 is shown in Table 2. ICI 182,780 demonstrates a pure antiestrogen profile in most tissues tested and is now FDA approved as an adjuvant chemotherapeutic to treat Tamoxifen-resistant tumors. The mechanism of action of this SERM appears to differ significantly from others. In contrast to other SERMs, ICI 182,780 is known to block ER transcription coming from both AF-1 and AF-2 domains but does appear to exhibit estrogenic effects at AP-1 sites. ICI 182,780 also may impair ER dimerization and lead to a marked reduction in cellular concentrations of ER by disrupting nucleocytoplasmic shuttling.

Initial studies have demonstrated that ICI 182,780 can directly induce intracellular calcium rise (see FIG. 1), activate the phosphorylation of ERK (see FIG. 3) and potentiate the expression of antiapoptotic protein Bcl-2 (see FIG. 4) in primary hippocampal neurons, all of which have been associated with the neuroprotective mechanism elicited by 17 β-estradiol, and thus, indicating the agonist-like effect of ICI 182,780 in the brain. Furthermore, ratiometric fluorescent calcium-imaging analyses revealed that neurons pretreated with ICI 182,780 and then exposed to excitotoxic glutamate indicated an attenuation of the glutamate-induced rise in intracellular calcium which is also a mechanism through which estrogen has been shown to be neuroprotective (see FIG. 2). However, it has been shown ICI 182,780 does not cross the blood-brain-barrier. Therefore, its effectiveness at treating neurodegenerative diseases in vivo is likely to be limited. IC 164,384 is another antiestrogen, with a chemical structure similar to that of ICI 182,780. However, this molecule also does not appear to cross the blood brain barrier.

It is therefore an object of the invention to provide SERMs with improved activity, particularly SERMs that cross the blood brain barrier and preferentially function in the brain rather than other tissues, and methods of making and using thereof.

### SUMMARY

Selective estrogen receptor agonist and/or antagonist modulators (“modulators”), pharmaceutical compositions thereof, and methods for treating or preventing estrogen receptor-mediated disorders using such modulators are described herein. The estrogen receptor modulators described herein contain two general structural features: (1) a head moiety and (2) a tail moiety, with the head moiety being relatively hydrophilic and the tail moiety being relatively hydrophobic. The head moieties of the modulators generally contain a skeletal chemical structure including (1) a steroidal structure, (2) a flavonoid structure, (3) an isoflavonoid structure or (4) a dibenzalkanal structure. The head moieties generally have at least two hydrophilic groups configured approximately at opposing ends of the head structural moiety. In one embodiment, at least one of these hydrophilic groups is a hydroxyl group. In another embodiment, two hydroxyl groups are present at roughly opposite ends of the head moiety.

The roughly opposing hydrophilic groups allow the head moiety to interact with polar amino acid side chains located within the binding pocket of an estrogen receptor. The predicted hydrogen bonding interactions of the head moiety with the ligand binding pocket of the estrogen receptor are expected to generate high binding affinity of the modulators to an estrogen receptor. The tail moiety generally contains a relatively long hydrocarbon chain of about 10 to 30 carbons, optional substituted with one or more substituents. The carbon chains may contain one or more heteroatoms, such as oxygen, nitrogen, sulphur, and combinations thereof. It is expected that the tail moiety of the modulator ligands interacts with coactivator sites located on an estrogen receptor. Optional substitution of the hydrocarbon chain of the tail moiety should yield tissue specific selectivity of the estrogen receptor modulator and allow modulators to cross the blood-brain-barrier.

These estrogen receptor modulators possess activity in modulating estrogen receptor activity. In particular, the modulators generally possess mixed estrogen receptor agonist/antagonist activities in distinct tissue, and specifically, possess agonist effects in the brain and antagonist effects in breast and uterine tissue. Thus, the modulators have utility in preventing or treating estrogen receptor-mediated disorders such as osteoporosis, breast and endometrial cancers, atherosclerosis, and Alzheimer's disease and other neurodegenerative diseases and related disorders. These modulators may also be used to treat one or more symptoms associated with menopause, such as hot flushes/flashes.

### BRIEF DESCRIPTION OF THE FIGURES

FIGS. 1A and 1B are graphs showing the intracellular calcium rise (in nM) in rat primary hippocampal neurons as a function of time (minutes) in response to 17β-estradiol (FIG. 1A) and ICI 182,780 (FIG. 1B).

FIGS. 2A and 2B are graphs showing that the SERM ICI 182,780 potentiates the physiological glutamate-induced rise in calcium ion concentration (nM) versus time (minutes) (FIG. 2A) and attenuates the excitotoxic glutamate induced rise in calcium ion concentration (nM) bersus time (minutes) (FIG. 2B) in neurons.

FIG. 3 is a graph showing the effect of known SERMs on relative Erk 2 phosphorylation in rat hippocampal neurons.

FIG. 4 is a graph showing the effect of known SERMs on relative Bcl-2 expression in rat hippocampal neurons.

FIG. 5 is a representation of an estrogen receptor modulator containing a “head” moiety substituted with two hydroxyl groups and a “tail” moiety, which is predicted via modeling to engender strong hydrogen-bond interactions with amino acid residues Glu353 and His524 with the ligand binding pocket of the estrogen receptor.

FIGS. 6A-6C are computer models of the complex of human ERα ligand binding domain (LBD) with ICI 164,384 (FIG. 6A), E² (FIG. 6B) and DPN-ICI (FIG. 6C). FIGS. 6A-6C also shows the intermolecular energies between the compounds and human ERα ligand binding domain (LBD).

FIGS. 7A-7D show the three-dimensional structures of ICI 164,384 (FIG. 7A)); NS1 (FIG. 7B)); Gen-ICI (FIG. 7C); and DPN-ICI (FIG. 7D).

FIGS. 8A and 8B are computer models of the complex structure of human ERα ligand binding domain (LBD) with ICI 164,384 (FIG. 8A) and NS1 (FIG. 8B). Computer modeling of the complex structure of human ERα LBD with ICI 164,384 and NS1 shows NS1 has similar binding mode and orientation as ICI 164,384 in ERα LBD. In addition, NS1 engender strong hydrogen-bond interactions with amino acid residues Glu353 and His524 as ICI 164,384 does. The modeling is generated by homology modeling based on the crystallographic complex structure of ICI 164,384 with rat ERβ (PDB code: 1HJ1) and by molecular docking with automatic computer docking program GOLD.

FIGS. 9A and 9B are graphs showing NS1's binding affinity (fluorescence polarization, mP) to both estrogen receptor α (FIG. 9A) and β (FIG. 9B) as a function of the concentration of NS1 (M).

FIGS. 10A and 10B are graphs showing NS1's neuroprotective ability against excitotoxic glutamate challenge as a function of the concentration of NS1 (nM). FIG. 10A is a graph showing the percent LDH release as a function of the concentration (nM) of NS1. FIG. 10B is a graph showing the percent calcein AM staining as a function of the concentration (nM) of NS1.

FIGS. 11A and 11B are graphs showing NS1's regulation of estrogenic mechanisms leading to neuroprotective outcomes, i.e. activation of ERK (FIG. 11A) and activation of AKT (FIG. 11B) signaling pathways.

FIGS. 12A and 12B are graphs showing NS1's regulation of estrogenic mechanisms leading to neuroprotective outcomes, i.e. upregulation of anti-apoptotic protein Bcl-2 (FIG. 12A) and upregulation of anti-apoptotic protein Bcl-xL (FIG. 12B).

FIGS. 13A-E are graphs showing the neuroprotective efficacy of NS2 (FIG. 13A); NS1-1 (FIG. 13B); NS1-2 (FIG. 13C); NS1-3 (FIG. 13D); and NS1-4 (FIG. 13E) against glutamate-induced neurotoxicity in rat primary hippocampal neurons as a function of time and concentration (nM).

FIG. 14A-E are graphs showing the competition binding curves for ERα and ERβ (molar concentration vs. fluorescence polarization (mP)) for NS2 (FIG. 14A); NS1-1 (FIG. 14B); NS1-2 (FIG. 14C); NS1-3 (FIG. 14D); and NS1-4 (FIG. 14E).

FIGS. 15A-C are graphs showing the percent increase in MCF-7 cell proliferation versus concentration for 17β-estradiol (FIG. 15A), ICI 182,780 (FIG. 15B), and NS1 (FIG. 15C).

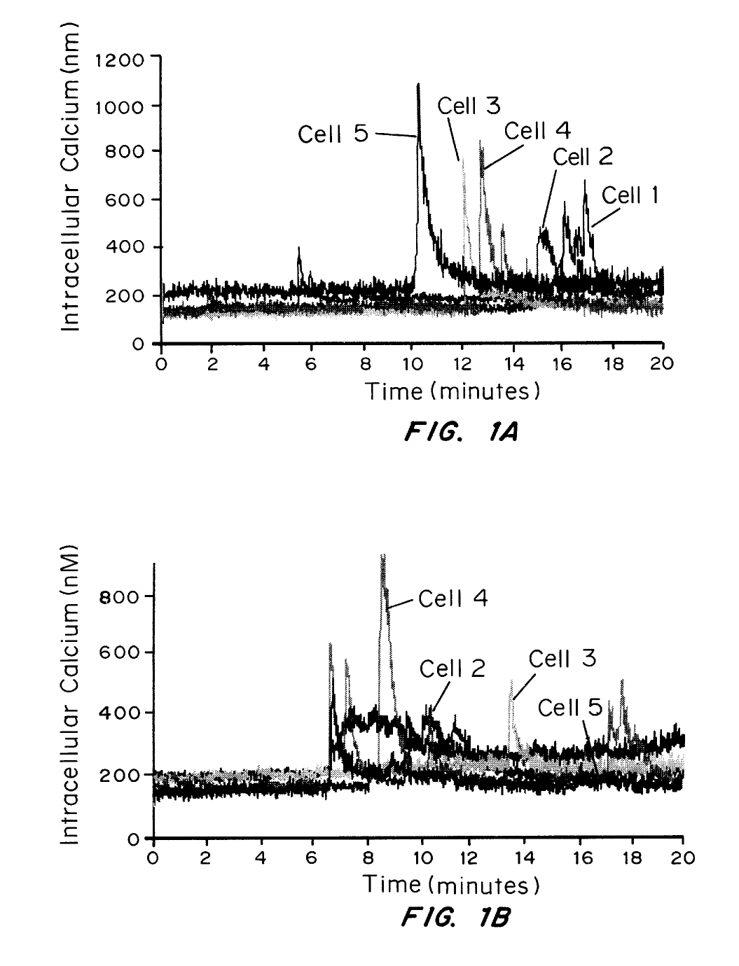

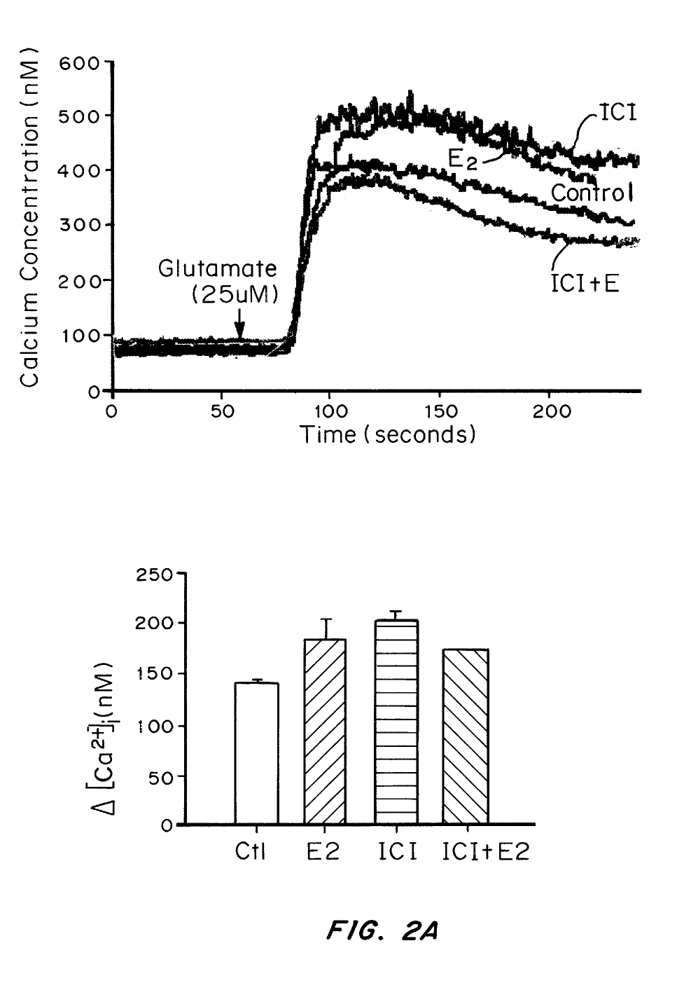

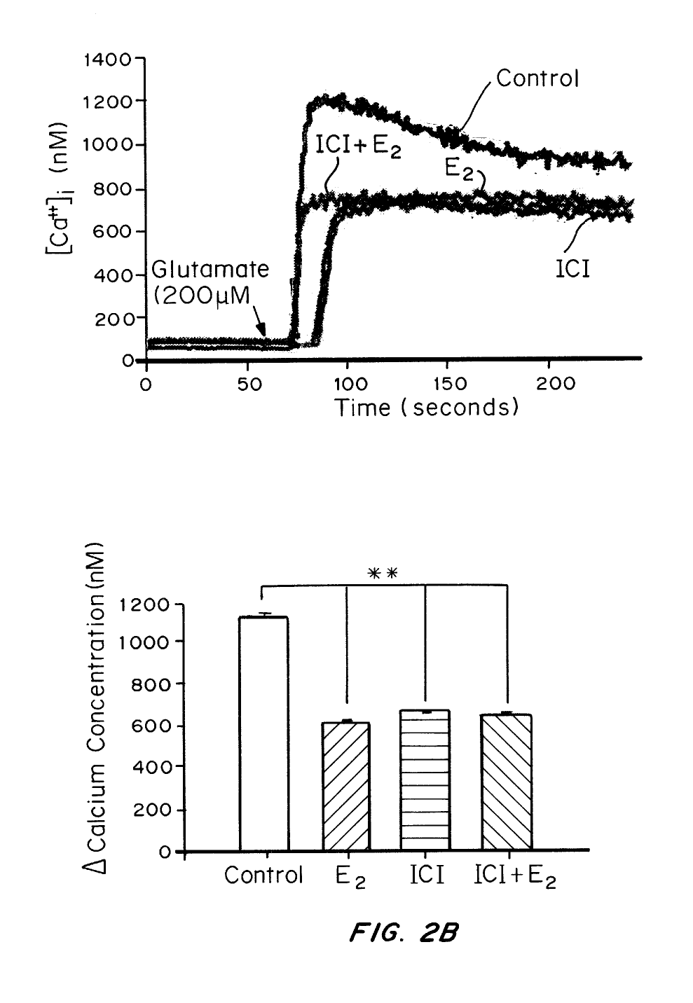

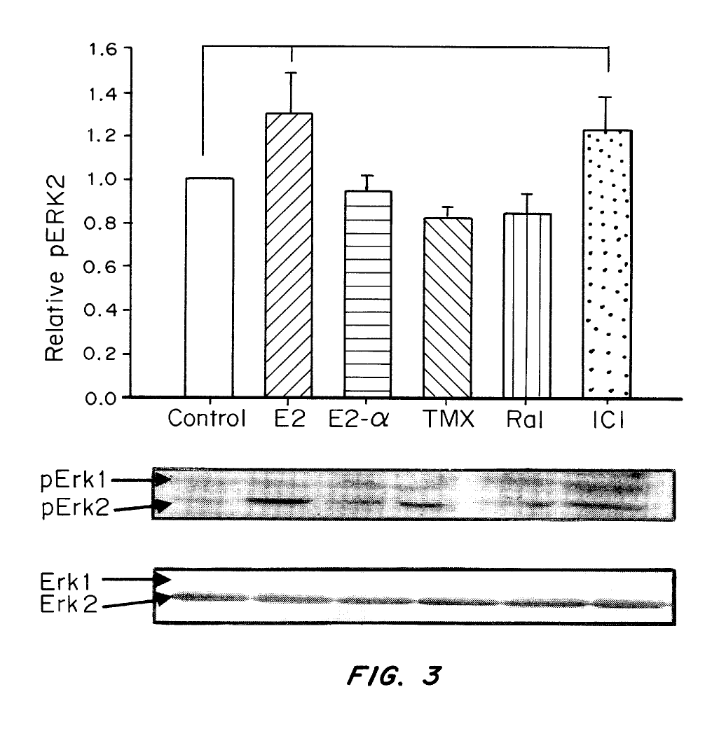

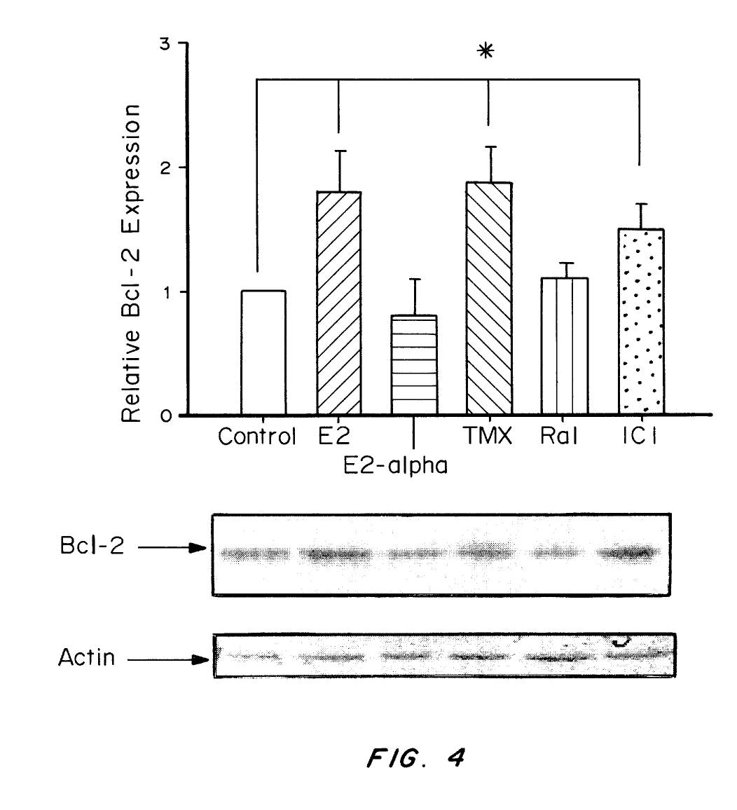

### DETAILED DESCRIPTION

Estrogen receptor modulators, pharmaceutical compositions thereof, and methods of use thereof are described herein. The estrogen receptor modulators include compounds that exhibit estrogen receptor antagonist activity or mixed selective estrogen receptor antagonist and agonist activity, i.e., SERM activity, in specific tissue types. These compounds are useful for treating and/or preventing a variety of diseases, particularly estrogen receptor-mediated diseases and disorders, such as osteoporosis, menopause, breast and endometrial cancers, arthroscleroses and Alzheimer's disease.

### I. DEFINITIONS

“Estrogen Receptor”, as used herein, refers to any protein in the nuclear receptor gene family that binds estrogen, including, but not limited to, any isoforms, including isoforms not known to date. More particularly, the present disclosure relates to estrogen receptor(s) for human and non-human mammals (e.g., animals of veterinary interest such as horses, cows, sheep, and pigs, as well as household pets such as cats and dogs). Human estrogen receptors include, but are not limited to, the alpha- and beta-isoforms (referred to herein as “ERα” and “ERβ”) in addition to any additional isoforms as recognized by those of skill in the biochemistry and molecular biology arts.

“Estrogen Receptor Modulator”, as used herein, refers to a compound that can act as an estrogen receptor agonist or antagonist of an estrogen receptor or estrogen receptor isoform having an IC₅₀ or EC₅₀ with respect to ERα, ERβ and/or other estrogen receptor isoforms of no more than about 50 μM as determined using the ERα, and/or ERβ transactivation assay described below. More typically, estrogen receptor modulators have IC₅₀ or EC₅₀ values (as agonists or antagonists) of not more than about 10 μM. Representative compounds are predicted to exhibit agonist or antagonist activity viz. an estrogen receptor. Compounds preferably exhibit an antagonist or agonist IC₅₀ or EC₅₀ with respect to ERα and/or ERβ of about 10 μM, more preferably, about 500 nM, even more preferably about 1 nM, and most preferably, about 500 pM, when measured in the ERα and/or ERβ transactivation assays. “IC₅₀” is that concentration of compound which reduces the activity of a target (e.g., ERα or ERβ) to half-maximal level. “EC₅₀” is that concentration of compound which provides half-maximum effect.

“Selective Estrogen Receptor Modulator” (or “SERM”), as used herein, refers to a compound that exhibits activity as an agonist or antagonist of an estrogen receptor (e.g., ERα, ERβ or other estrogen receptor isoform) in a tissue-dependent manner. Thus, as will be apparent to those of skill in the biochemistry, molecular biology and endocrinology arts, compounds that function as SERMs can act as estrogen receptor agonists in some tissues, e.g., bone, brain, and/or cardiovascular, and as antagonists in other tissue types, e.g., the breast and/or uterine tissue, A NeuroSERM is a subset of the SERM embodiments that exhibits activity as an agonist of an estrogen receptor in brain tissue and exhibits activity as an antagonist of an estrogen receptor in other tissue, e.g. breast and/or uterine tissue. The words “ligand” and the word “compound” are two words used to describe the estrogen receptor modulators. The word “ligand” is used generally in reference to the binding properties of the estrogen receptor modulators to the lipid binding domain or pocket of the estrogen receptor. The word “compound” is used generally to denote the molecule itself without particular reference to its binding properties. However, these two words may be used interchangeably.

“Optionally substituted”, as used herein, refers to the replacement of hydrogen with a monovalent or divalent radical. Suitable substituents include, but are not limited to, hydroxyl, nitro, amino, imino, cyano, halo, thio, thioamido, amidino, oxo, oxamidino, methoxamidino, imidino, gumidino, sulfonamido, carboxyl, formyl, loweralkyl, cycloalkyl, heterocycloalkyl, halo-loweralkyl, loweralkoxy, halo-loweralkoxy, loweralkoxyalkyl, alkylcarbonyl, aryl, heteroaryl, arylcarbonyl, aralkylcarbonyl, heteroarylcarbonyl, heteroaralkylcarbonyl, alkylthio, aminoalkyl, and cyanoalkyl. The substituent can itself be substituted. The group substituted onto the substitution group can be, for example, carboxyl, halo; nitro, amino, cyano, hydroxyl, loweralkyl, loweralkoxy, aminocarbonyl, —SR, thioamido, —SO₃H, —SO₂R or cycloalkyl, where R is typically hydrogen, hydroxyl or loweralkyl. When the substituted substituent includes a straight chain group, the substitution can occur either within the chain (e.g., 2-hydraxypropyl, 2-aminobutyl) or at the chain terminus (e.g., 2-hydroxyethyl, 3-cyanopropyl). Substituted substituents can be straight chain, branched or cyclic arrangements of covalently bonded carbon or heteroatoms.

“Loweralkyl”, as used herein, refers to branched or straight chain alkyl groups comprising one to ten carbon atoms that independently are unsubstituted or substituted, e.g., with one or more halogen, hydroxyl or other groups, Examples of loweralkyl groups include, but are not limited to, methyl, ethyl, propyl, isopropyl, n-butyl, t-butyl, n-hexyl, neopentyl, trifluoromethyl, pentafluoroethyl. Examples of substituted loweralkyl groups include the optionally substitutions given above.

“Alkenyl”, as used herein, refers to a divalent straight chain or branched chain saturated aliphatic radical having from 10 to 30 carbon atoms. “Alkenyl” refers herein to straight chain, branched, or cyclic radicals having one or more double bonds and from 10 to 30 carbon atoms. “Alkynyl” refers herein to straight chain, branched, or cyclic radicals having one or more triple bonds and from 10 to 30 carbon atoms. An alkylenyl group, alkenyl group and/or an alkynyl group can be further optionally substituted to yield a straight chain, branched, or cyclic radical that comprises more than 10 to 30 carbon atoms.

“Halo”, as used herein, refers to a halogen radical, e.g., fluorine, chlorine, bromine, or iodine.

“Aryl”, as used herein, refers to monocyclic and polycyclic aromatic groups, or fused ring systems having at least one aromatic ring, having from 3 to 14 backbone carbon atoms. Examples of aryl groups include without limitation phenyl, naphthyl, dihydronaphthyl, tetrahydronaphthyl.

“Aralkyl”, as used herein, refers to an alkyl group substituted with an aryl group. Typically, aralkyl groups employed in compounds have from 1 to 6 carbon atoms incorporated within the alkyl portion of the aralkyl group. Suitable aralkyl groups employed in compounds include, for example, benzyl, picolyl.

“Heteroaryl”, as used herein, refers to aryl groups having from one to four heteroatoms as ring atoms in an aromatic ring with the remainder of the ring atoms being aromatic or non-aromatic carbon atoms. When used in connection with aryl substituents, the term “poly cyclic” refers herein to fused and non-fused cyclic structures in which at least one cyclic structure is aromatic, such as, for example, benzodioxozolo, naphthyl. Exemplary heteroaryl moieties employed as substituents in compounds include pyridyl, pyrimidinyl, thiazolyl, indolyl, imidazolyl, oxadiazolyl, tetrazolyl, pyrazinyl, triazolyl, thiophenyl, furanyl, quinolinyl, purinyl, benzothiazolyl, benzopyridyl, and benzimidazolyl.

“Amino”, as used herein, refers to the group NH₂. The term “loweralkylamino” refers herein to the group —NRR′ where R and R′ are each independently selected from hydrogen or loweralkyl. The term “arylamino” refers herein to the group —NRR′ where R is aryl and R′ is hydrogen, loweralkyl, aryl, or aralkyl. The term “aralkylamino” refers herein to the group —NRR′ where R is aralkyl and R′ is hydrogen, loweralkyl, aryl, or aralkyl. The terms “heteroarylamino” and “heteroaralkylamino” are defined by analogy to arylamino and aralkylamino.

The term “aminocarbonyl”, as used herein, refers to the group —C(O)—NH₂. The terms “loweralkylaminocarbonyl”, “arylaminocarbonyl”, “aralkylaminocarbonyl”, “heteroarylaminocarbonyl” and “heteroarylaminocarbonyl” refer to —C(O)NRR′ where R and R′ independently are hydrogen and optionally substituted loweralkyl, aryl, aralkyl, heteroaryl, and heteroaralkyl respectively by analogy to the corresponding terms above.

The term “thio” refers to —SH. The terms “loweralkylthio”, “arylthio”, “heteroarylthio”, “cycloalkylthio”, “cycloheteroalkylthio”, “aralkylthio”, “heteroaralkylthio′, “(cycloalkyl)alkylthio, and “(cycloheteroalkyl)alkylthio, where R is optionally substituted with loweralkyl, aryl, heteroaryl, cycloalkyl, cycloheteroalkyl, aralkyl, heteroaryl, (cycloalkyl)alkyl, and (cycloheteroalkyl)alkyl respectively.

The term “sulfonyl” refers herein to the group —SO₂—. The terms “loweralkylsulfonyl”, “arylsulfonyl”, “heteroarylsulfonyl”, “cycloalkylsulfonyl”, “heteroalkylsulfonyl”, “aralkylsulfonyl”, “heteroaralkylsulfonyl”, (cycloalkyl)alkylsulfonyl”, and “(cycloheteroalkyl)alkylsulfonyl” refer to —SO₂R where R is optionally substituted loweralkyl, aryl, heteroaryl, cycloalkyl, cycloheteroalkyl, aralkyl, heteroaralkyl, (cycloalkyl)alkyl, and (cycloheteroalkyl)alkyl respectively.

The term “sulfinyl” refers herein to the group —SO—. The terms loweralkylsulfinyl”, “arylsulfinyl”, “heteroarylsulfinyl”, “cycloalkylsulfinyl”, “cycloheteroalkylsulfinyl”, “aralkylsulfinyl”, “heteroaralkylsulfinyl”, “(cycloalkyl)alkylsulfinyl”, and “(cycloheteroalkyl)alkylsulfinyl” refer to —SOR where R is optionally substituted loweralkyl, aryl, heteroaryl, cycloalkyl, cycloheteroalkyl, aralkyl, heteroaralkyl, (cycloalkyl)alkyl, and (cycloheteroalkyl)alkyl respectively.

“Formyl” refers to —C(O)H.

“Carboxyl” refers to —C(O)OH.

“Carbonyl” refers to the divalent group —C(O)—. The terms “loweralkylcarbonyl”, “arylcarbonyl”, “heteroarylcarbonyl”, “cycloalkylcarbonyl”, “cycloheteroalkylcarbonyl”, “aralkylcarbonyl”, “heteroaralkylcarbonyl”, “(cycloalkyl)alkylcarbonyl”, and “(cycloheteroalkyl)alkylcarbonyl” refer to —C(O)R, where R is optimally substituted loweralkyl, aryl, heteroaryl, cycloalkyl, cycloheteroalkyl, aralkyl, heteroaralkyl, (cycloalkyl)alkyl, and (cycloheteroalkyl)alkyl respectively.

“Thiocarbonyl” refers to, the group —C(S)—. The terms “loweralkylthiocarbonyl”, “arylthiocarbonyl”, “heteroarylthiocarbonyl”, “cycloalkylthiocarbonyl”, “cycloheteroalkylthiocarbonyl”, “aralkyldiocarbonyloxlthiocarbonyl”, “heteroaralkylthiocarbonyl”, “(cycloalkyl)alkylthiocarbonyl”, “(cycloheteroalkyl)alkylthiocarbonyl” refer to —C(S)R, where R is optionally substituted loweralkyl, aryl, heteroaryl, cycloalkyl, cycloheteroalkyl, aralkyl, heteroaralkyl, (cycloalkyl)alkyl, and (cycloheteroalkyl)alkyl respectively.

“Carbonyloxy” refers generally to the group —C(O)—O—. The terms “loweralkylcarbnyloxy”, “arylcarbonyloxy”, “heteroarylcarbonyloxy”, “cycloalkylcarbonyloxy”, cycloheteroalkylcarbonyloxy”, “aralkylcarbonyloxy”, “heteroaralkylcarbonyloxy”, “(cycloalkyl)alkyl cabonyloxy,” (cycloheteroalkyl)alkylcarbonyloxy” refer to —C(O)OR, where R is optionally substituted loweralkyl, aryl, heteroaryl, cycloalkyl, cycloheteroalkyl, aralkyl, heteroaralkyl, (cycloalkyl)alkyl, and (cycloheteroalkyl)alkyl respectively,

“Oxycarbonyl” refers to the group —O—C(O)—, The terms “loweralkyloxycarbonyl”, “aryloxycarbonyl”, “heteroaryloxycarbonyl”, “cycloalkyloxycarbonyl”, “cycloheteroalkyloxycarbonyl”, “aralkyoxycarbonyloxloxycarbonyl”, “heteroaralkyloxycarbonyl”, “(cycloalkyl)alkyloxycarbonyl”, “(cycloheteroalkyl)alkyloxycarbonyl” refer to -0-C(O)R, where R is optionally substituted loweralkyl, -1, hetemq1, cycloalkyl, cycloheteroalkyl, aralkyl, heteroaralkyl, (cycloalkyl)alkyl, and (cycloheteroalkyl)alkyl respectively.

“Carbonylamino” refers to the group —NH—C(O)—. The terms “loweralkylcarbonylamino”, “arylcarbonylamino”, “heterocarbonylamino”, “cycloalkylcarbonylamino”, “cycloheteroalkylcarbonylamino”, “aralkylcarbonylamino”, “heteroaralkylcarbonylamino”, “(cycloalkyl)alkylcarbonylamino”, and “(cycloheteroalkyl)alkylcarbonylamino” refer to —NH—C(O)R, where R is optionally substituted loweralkyl, aryl, heteroaryl, cycloalkyl, cycloheteroalkyl, aralkyl, heteroaralkyl, (cycloalkyl)alkyl, or (cycloheteroalkyl)alkyl respectively. In addition, the present disclosure includes N-substituted carbonylamino (—NR′C(O)R), where R′ is optionally substituted loweralkyl, aryl, heteroaryl, aralkyl, or heteroaralkyl and R retains the previous definition.

As used herein, the term “amidino” refers to the moieties R—C(═N)—NR′— (the radical being at the “N¹” nitrogen) and R(NR′)C═N— (the radical being at the “N²” nitrogen), where R and R′ can be hydrogen, loweralkyl, aryl, or loweraralkyl.

The term “imino” refers to the group —C(—NR)—, where R can be hydrogen or optionally substituted loweralkyl, aryl, heteroaryl, or heteroaralkyl respectively. The terms “iminoloweralkyl”, “iminocycloalkyl”, “iminocycloheteroalkyl” “iminoaralkyl”, “iminoheteroaralkyl”, “cycloalkyl)iminoalkyl”, (cycloiminoalkyl)alkyl”, “(cycloiminohetero)alkyl, and “(cyloheteroalkyl)iminoalkyl, optionally substituted loweralkyl, cycloalkyl, cycloheteroalkyl, aralkyl, heteroaralkyl, (cycloalkyl)acyl, and (cycloheteroalkyl)alkyl groups that include an imh6 group, respectively.

The term “oximino” refers to the group —C(═NOR)—, where R can be hydrogen (“hydroximino”) or optionally substituted loweralkyl, aryl, heteroaryl, or heteroalkyl respectively. The terms “oximinoloweralkyl”, “oximinocycloalkyl”, “oximinocycloheteroalkyl”, “oximinoaralkyl”, “oximinoheteroalkyl”, “(cycloalkyl)oximinoalkyl” “(cyclooximinoalkyl)alkyl”, “(cyclooximinoheteroalkyl)alkyl”, and (cycloheteroalkyl)oximinoalkyl refer to optionally substituted loweralkyl, cycloalkyl, cycloheteroalkyl, aralkyl, heteroaralkyl, (cycloalkyl)alkyl, and (cycloheteroalkyl)alkyl groups that include an oximino group, respectively.

The term “methylene” as used herein refers to an unsubstituted, monosubstituted, or disubstituted carbon atom having a formal sp3 hybridization (is., —CRR′—, where R and R′ are hydrogen or independent substituents).

The term “methine” used herein refers to an unsubstituted or carbon atom having a formal sp2 hybridization (i.e., —CR═ or ═CR—, where R is hydrogen a substituent.

As used herein, the term “analogue” refers closely related, typically synthetic members of a chemotype—a family of molecules that demonstrate a unique core structure or scaffold—with minor chemical modifications that might show improved target-binding affinity and potency compared with the original natural lead compound.

As used herein, the term “derivative” refers to a compound that is formed from a similar compound or a compound that can be imagined to arise from another compound, if one atom is replaced with another atom or group of atoms.

### II. SELECTIVE ESTROGEN RECEPTOR MODULATORS AND FORMULATIONS THEREOF

A. Compounds

Preferred embodiments, referred to as NeuroSERMs™, promote neurotrophism and neuroprotection in brain tissue. These NeuroSERMs™ represent a subset of the estrogen receptor modulators ligands/compounds described herein. NeuroSERMs™ can cross the blood-brain-barrier and exert estrogen receptor agonist-like effects in the brain. The estrogen receptor modulators can be used to prevent and/or treat neurological diseases, particularly those diseases associated with neurodegeneration, such as Alzheimer's disease. The modulators should also be useful for treating and/or preventing other estrogen receptor mediated diseases and disorders, such as osteoporosis, menopause, breast and endometrial cancers and atherosclerosis.

1. Compounds Containing “Head” and “Tail” Moieties

In one embodiment, the estrogen receptor modulator compounds contain a relatively hydrophilic “head” moiety and a relatively hydrophobic “tail” moiety. Predictive algorithms reveal that when the preferred estrogen receptor modulator compounds bind to an estrogen receptor, the head moiety of the modulator centrally docks within the ligand-binding pocket of the estrogen receptor with the tail moiety protruding from the ligand binding pocket. This predicted binding motif for the head moiety of the modulator compounds engenders strong interactions with vicinal amino acid residues and yields high predicted binding affinities. The tail moiety of estrogen receptor modulators is predicted to bind along or interact with a coactivator recruitment site of an estrogen receptor and is the moiety that may be responsible for tissue selectivity of these compounds.

The positive effects of estrogen (17-β-estradial) on neuronal outgrowth and survival that have been observed in vitro and in vivo in the brain are counterbalanced by the negative side effects of estrogen in the reproductive tissues. Thus, in one embodiment, the NeuroSERMs are estrogen receptor modulators that exhibit agonist effects in brain tissue. The NeuroSERMs may also exhibit antagonist effects in non-brain tissue, such as uterus and breast, or the NeuroSERMs may be relatively neutral in their behavior in non-brain tissue, i.e., the NeuroSERMs may not substantially exhibit agonist or antagonist effects in non-brain tissue. These latter NeuroSERMs are generally ERβ selective, as reproductive tissue generally is not found to express ERβ.

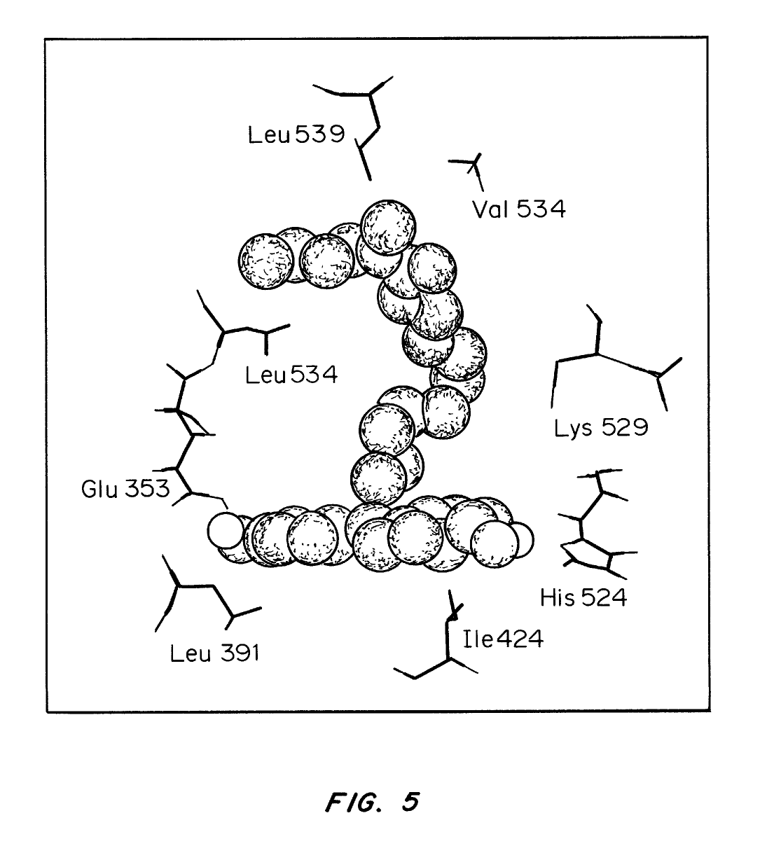

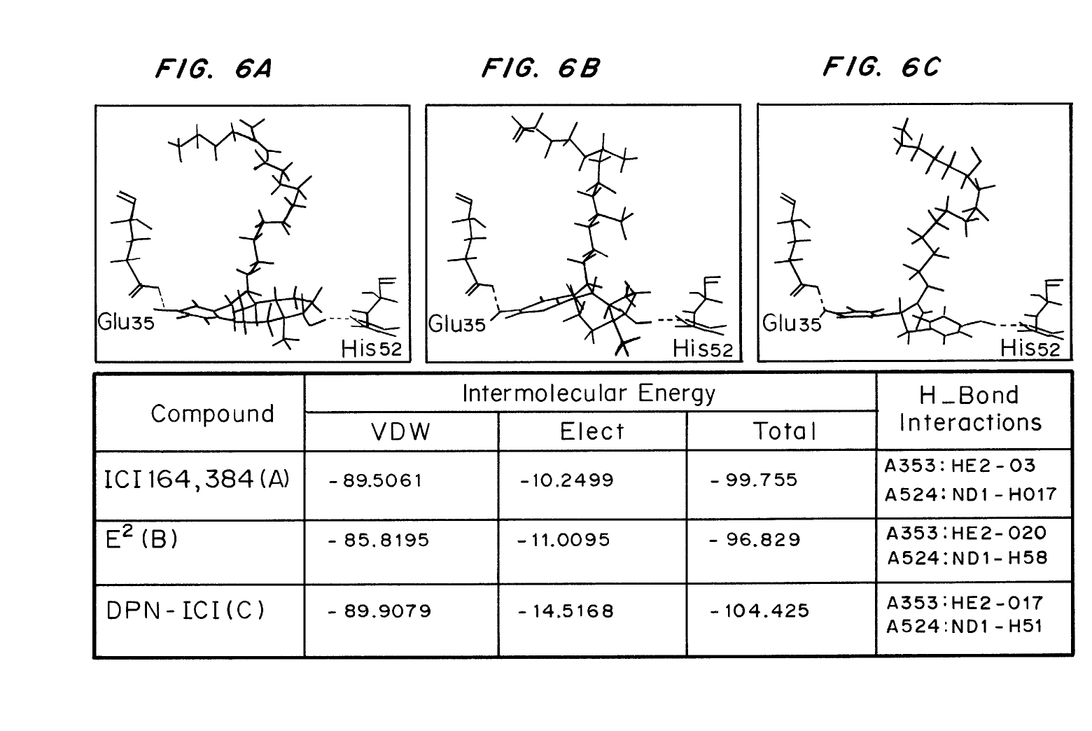

A series of NeuroSERMs have been designed and synthesized that are predicted to possess agonist effects in brain tissue, and moreover, are predicted to be found in the brain tissue of mammals, i.e., have the capacity to cross the blood-brain-barrier. FIG. 5 depicts an idealized ligand/compound associated with the ligand binding pocket, or domain, of an estrogen receptor. As depicted in FIG. 5, the head moiety binds within the ligand binding pocket of an isoform of the estrogen receptor, while the tail moiety protrudes out from this ligand binding pocket and is predicted to interact with a coactivator site on an isoform of the estrogen receptor. FIGS. 6A-C are computer models of the complex of human ERα ligand binding domain (LBD) with ICI 164,384 (FIG. 6A), E² (FIG. 6B) and DPN-ICI (FIG. 6C)

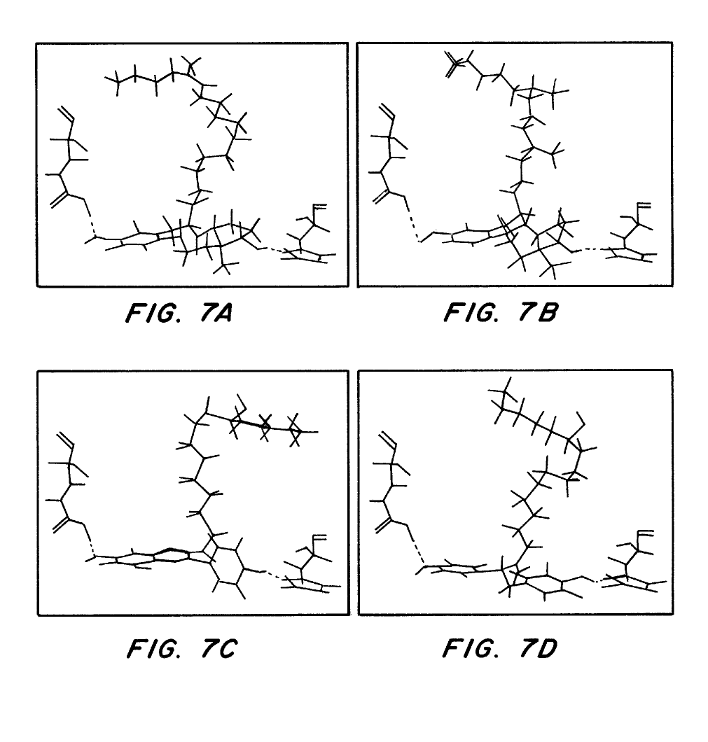

The NeuroSERMs described therein generally resemble the overall structure of ICI antagonist ligands, namely ICI 182,870 and ICI 164,384 in that these NeuroSERMs possess a head moiety and tail moiety (see FIGS. 7A-7D). The tail moiety is configured, in relation to the head moiety, in a manner that permits the tail moiety to protrude out from the ligand binding pocket. The tail moiety is designed to have chemical properties that allow the estrogen receptor modulator ligands/compounds to cross the blood-brain-barrier. These structural features of the NeuroSERMs are predicted to yield compounds that possess agonist effects, particularly in brain, and possess antagonist or neutral effects in other tissue, particularly breast and/or uterine tissue. The predicted binding energies for selected estrogen receptor modulator are shown in the table below.

TABLE 1
Predicted Binding Energies for Select
Estrogen Receptor Modulators

| | Intermolecular Energy (Human ERα) | | | |
|---|---|---|---|---|
| Compound | VDW | Elect | Total | H-bonds |
| ICI 164,384 | −89.5061 | −10.2499 | −99.755 | A353:HE2-O3* |
| (PDB: 1HJ1, | | | | A524:ND1- |
| Rat ERβ) | | | | HO17* |
| Estro-VitE | −85.8195 | −11.0095 | −96.829 | A353:HE2-O20* |
| | | | | A524:ND1-H58* |
| Gen-ICI | −92.1111 | −11.2858 | −103.397 | A353:HE2-O20* |
| | | | | A524:ND1-H48* |
| PPT-ICI | −100.38 | −8.3328 | −108.713 | A379:O-H60 |
| | | | | A525:O-H62 |
| DPN-ICI | −89.9079 | −14.5168 | −104.425 | A353:HE2-O17* |
| | | | | A524:ND1-H51* |

In one embodiment, the head moiety includes at least two hydrophilic groups, preferably located at approximately opposite sides of the head moiety. These hydrophilic groups are denoted as 4 H, however, this designation is meant to refer to any hydrophilic group. Suitable hydrophilic groups include, but are not limited to, hydroxyl, methoxy, thio, amino, halo, cyano, and combinations thereof. In a preferred embodiment, at least one hydrophilic group is a hydroxyl group. In another preferred embodiment, both hydrophilic groups are hydroxyl groups which are located at approximately opposite sides of the head moiety. Suitable head moieties include, but are not limited to, steroidal, flavonoid, isoflavonoid, dibenzalkanal, and 1,4-naphthoquinonyl moieties.

The head moieties may contain one or more additional substituents. The steroidal head moiety contains three groups, R1, R2 and R3 that are independently a hydrogen atom or other substituent or functional group. The flavonoidal head moiety contains seven groups, represented by R1, R2, R3, R1′, R2′, R3′ and R4′, that are independently a hydrogen atom or other substituent or functional group. The isoflavonoidal head moiety also contains seven groups, represented by R1, R2, R3, R1′, R2′, R3′ and R4′, that are independently a hydrogen atom or other substituent or functional group. Moreover, for both the flavonoidal and isoflavonoidal head moieties, the bond between positions 2 and 3 can be either a single or a double bond. The dibenzalkanal head moiety contains six groups, represented by R1, R2, R3, R1′, R2′ and R3′, that are independently a hydrogen atom or other substituent or functional group. The bond between the carbon at position 3 and the R5 group of the dibenzalkanal can be either a single bond or a double bond. Further, the R5 group found in the dibenzalkanal head moiety can be a hydrogen atom, a hydroxyl group, an amino group, a methyl group, a halide, a thio group, a methoxy group, a methylamino group or a methylthio group, if the bond between the carbon at position 3 and the group is a single bond. If, however, the bond between the carbon at position 3 and the R5 group is a double bond, then R5 is preferably an oxygen atom, a sulfur atom, an imino group or a methylimino group. In addition, preferably the number of carbon atoms in the main alkyl chain between the two benzene groups of the dibenz-alkanal skeletal chemical structure is 3, 4 or 5.

The head moieties can be substituted with one or more substituents or functional groups. Suitable substituents or functional groups include, but are not limited to, hydroxyl, nitro, amino, imino, cyano, halo, thio, thioamido, amidino, oxo, oxamidino, methoxamidino, imidino, gumidino, sulfonamido, carboxyl, formyl, loweralkyl, halo-loweralkyl, cycloalkyl, heterocycloalkyl, loweralkoxy, halo-loweralkoxy, loweralkoxyalkyl, alkylcarbonyl, aryl, heteroaryl, arylcarbonyl, aralkylcarbonyl, heteroarylcarbonyl, heteroaralkylcarbonyl, alkylthio, aminoalkyl, and cyanoalkyl. The substituent itself may also be substituted. The group substituted onto the functional group or substituent can be, for example, carboxyl, halo; nitro, amino, cyano, hydroxyl, loweralkyl, loweralkoxy, aminocarbonyl, —SR, thioamido, —SO₃H, —SO₂R or cycloalkyl, where R is typically hydrogen, hydroxyl or loweralkyl. When the substituted substituent includes a straight chain group, the substitution can occur either within the chain (e.g., 2-hydroxypropyl, 2-aminobutyl) or at the chain terminus (e.g., 2-hydroxyethyl, 3-cyanopropyl).

The five types of head moieties delineated above also have a tail moiety, represented by the R4 group. The tail moiety contains at least about 10 carbons. In one embodiment, the tail moiety contains a linear, straight chain portion of about 10 to about 30 carbon atoms, preferably from about 10 to about 20 carbon atoms; however, some of the carbon atoms may be replaced with other atoms, e.g. oxygen, nitrogen and sulphur. Preferably, no more than 5 carbon atoms are replaced with other atoms. The linear portion of the tail moiety may include one or more double bonds and/or triple bonds. Beyond the linear straight chain portion of the tail moiety, the tail moiety can have an overall branched or cyclic structure. The linear portion of the tail moiety, i.e., 10-30 carbons in a straight chain structure, can be optionally substituted with one or more functional groups or substituents other than hydrogen. These chemical groups maybe the same or different. For example, there can be 10 optionally substituted groups that are all the same, there can be 10 optionally substituted groups that are all different, or there can be variations in the substitution pattern that lies between these two extremes. An example of such a variation in between would be as follows: 1 substitution with group A, 2 substitutions with group B, 1 substitution with group C, 2 substitutions with group D and 4 substitutions with group E. The schemes shown below illustrate representative synthetic routes for the preparation of the compounds described herein.

Synthetic Routes Leading to the Compounds

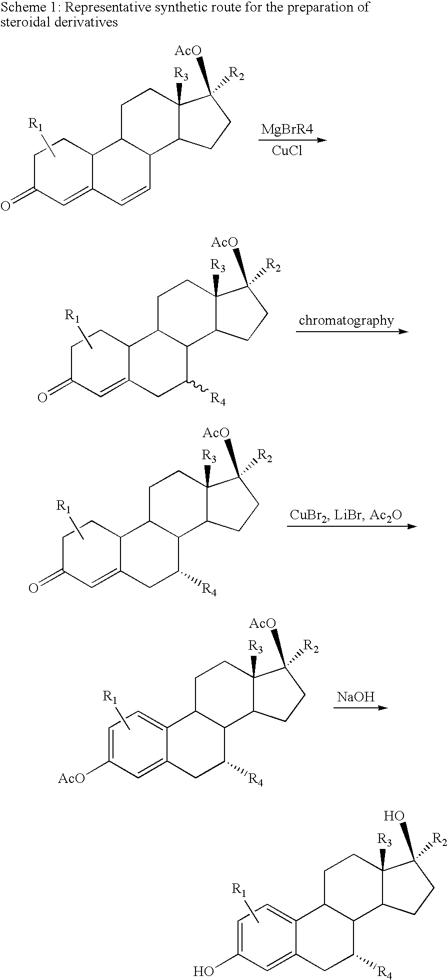

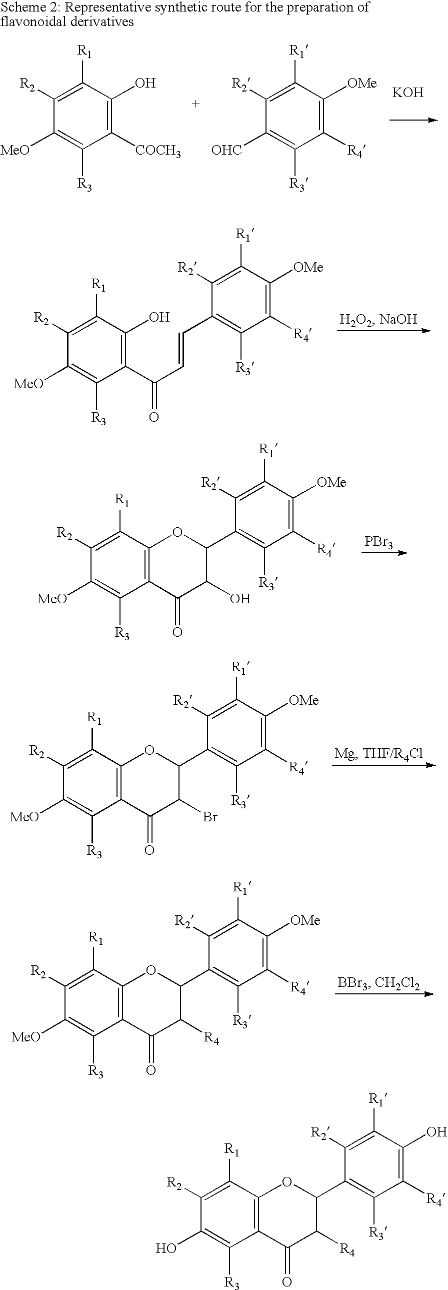

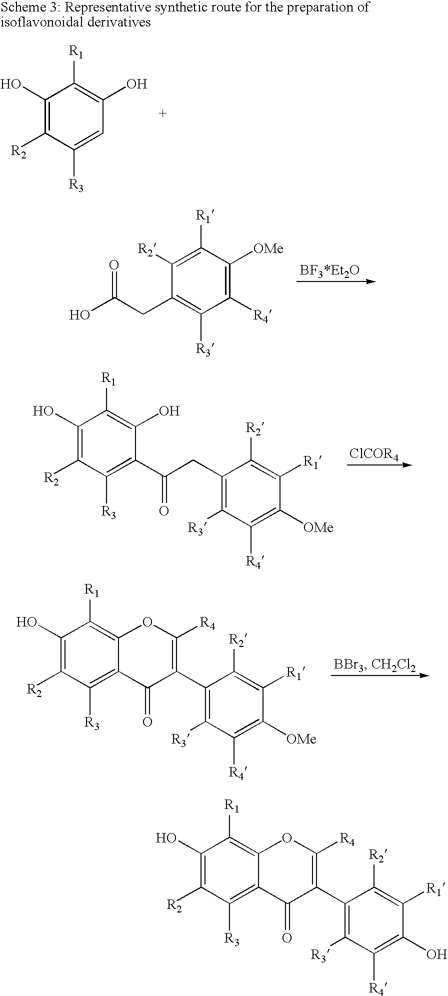

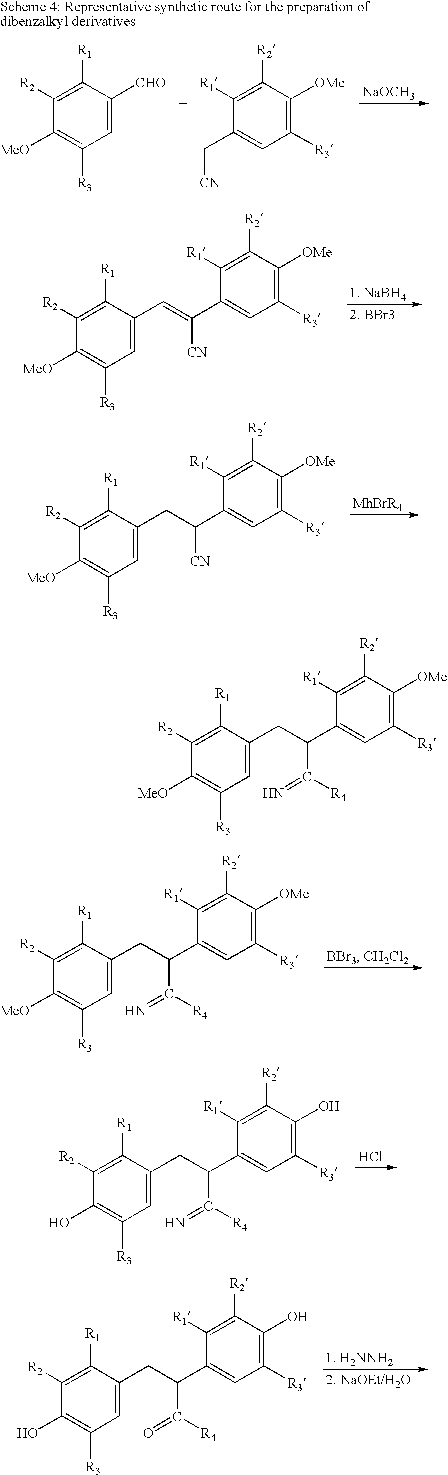

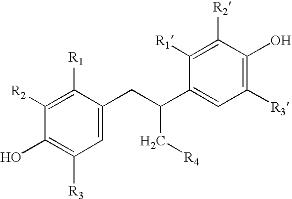

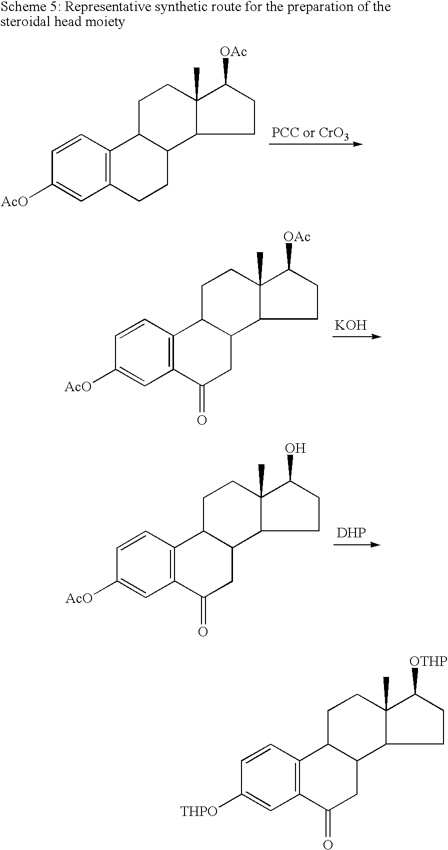

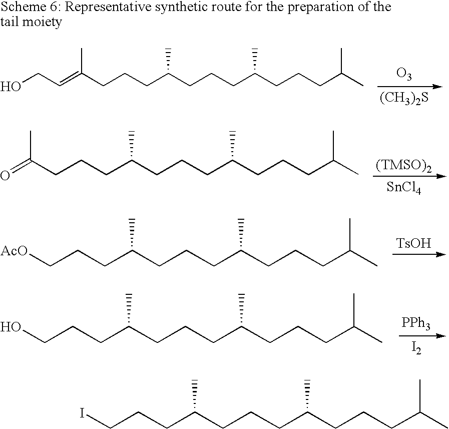

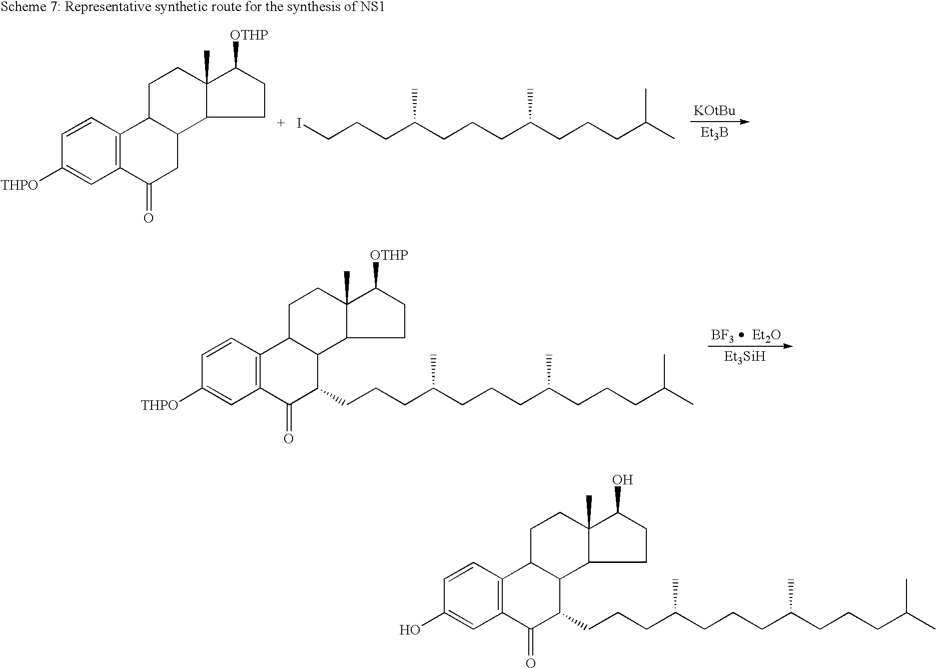

The complete synthesis of NS1 is described in Brinton et al., J. Med. Chem., 50, 4471-4481 (2007). The structure of NS1, analogs of NS1, and compounds with a new scaffold, NS2, are listed in Tables 2 and 3.

TABLE 2
Structures of Selective Estrogen Receptor Modulators.

| | |
|---|---|
| NS1 | 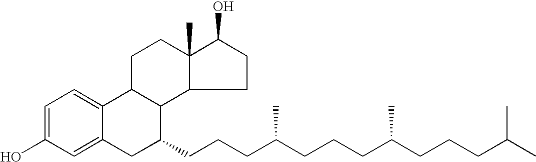 |
| NS1′ | 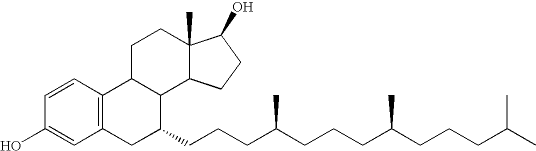 |
| NS1analogue 1 | 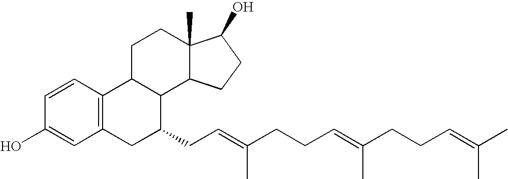 |
| NS1analogue 2 | 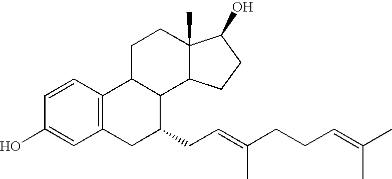 |
| NS1analogue 3 | 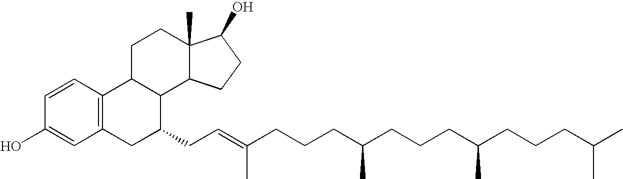 |
| NS1-1 | 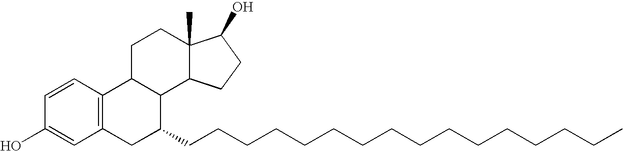 |
| NS1analogue 5 | 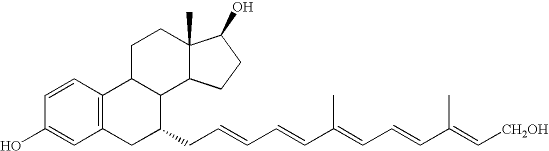 |
| NS2 | 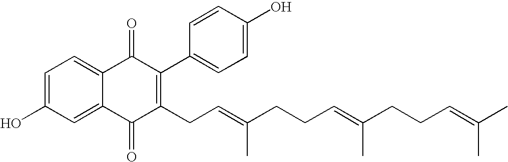 |
| NS 1-2 | 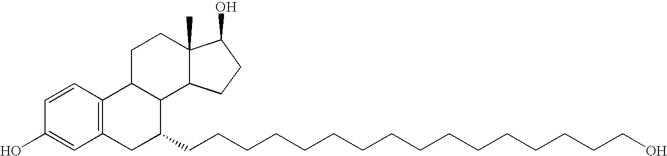 |
| NS 1-3 | 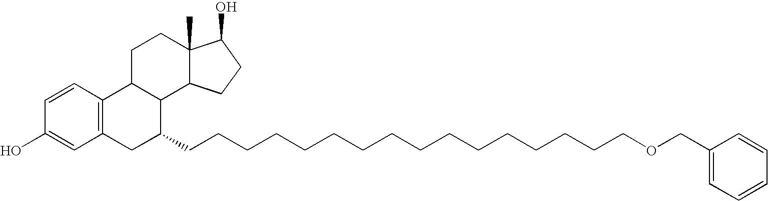 |
| NS 1-4 | 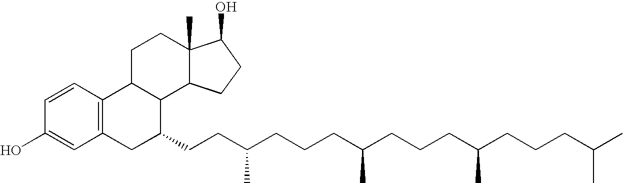 |

Some additional embodiments of estrogen receptor ligands/compounds are listed in Table 3. NS1 is listed as compound 1 in Table 3.

TABLE 3
Estrogen receptor compounds

| | |
|---|---|
| Vitamin E | 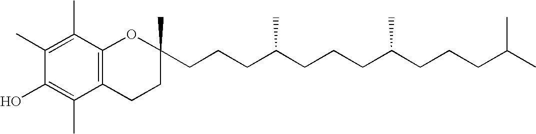 |
| CoenzymeQ | 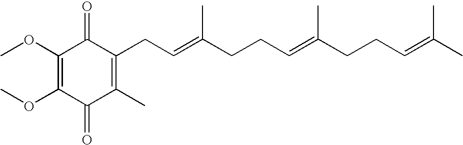 |
| ICI182,780 | 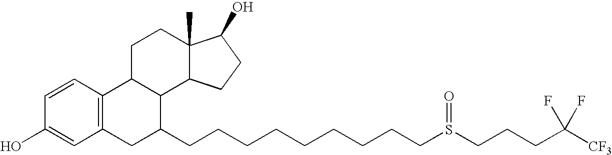 |
| ICI164,384 | 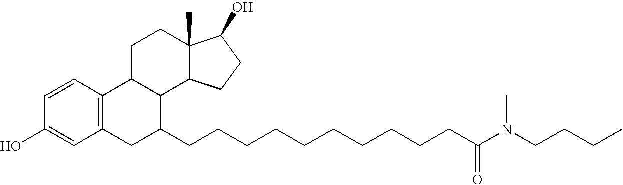 |
| Compd 1 | 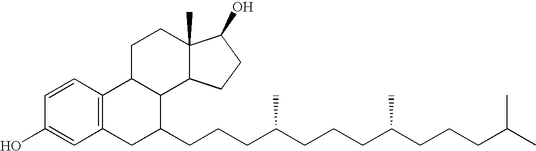 |
| Compd 2 | 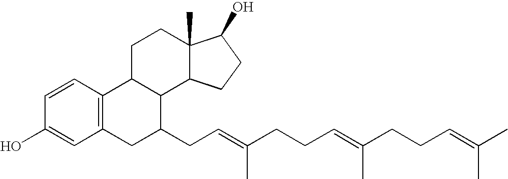 |
| Compd 3 | 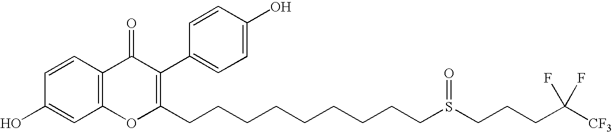 |
| Compd 4 | 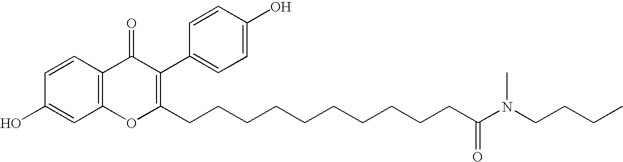 |
| Compd 5 | 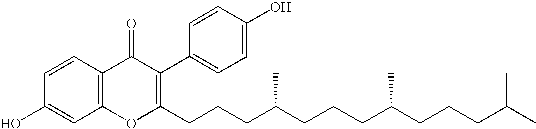 |
| Compd 6 | 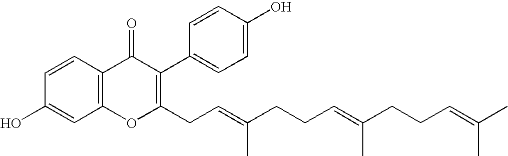 |
| Compd 7 | 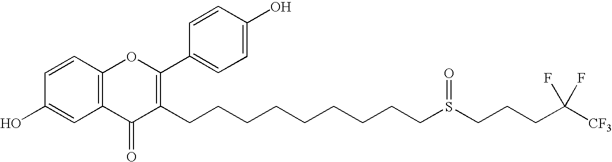 |
| Compd 8 | 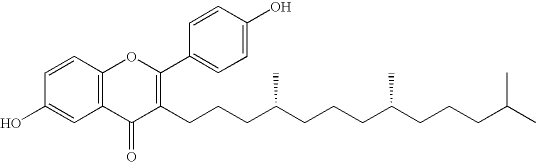 |
| Compd 9 | 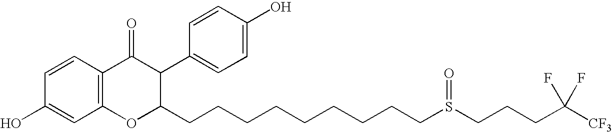 |
| Compd 10 | 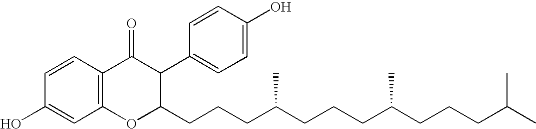 |
| Compd 11 | 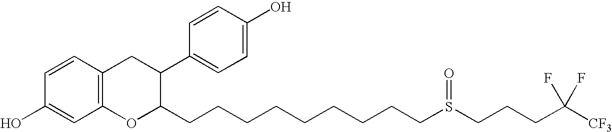 |
| Compd 12 | 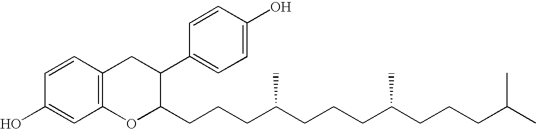 |
| Compd 13 | 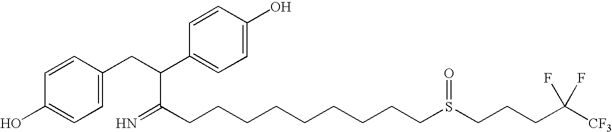 |
| Compd 14 | 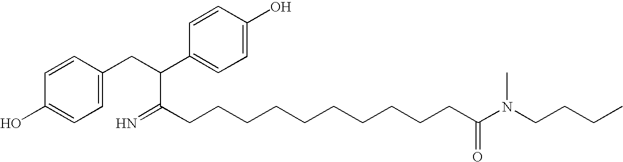 |
| Compd 15 | 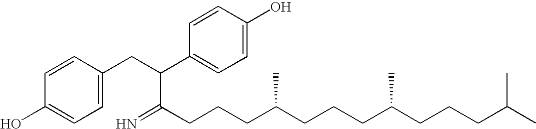 |
| Compd 16 | 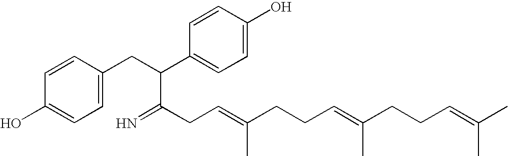 |
| Compd 17 | 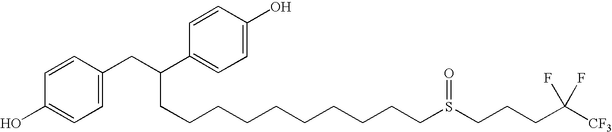 |
| Compd 18 | 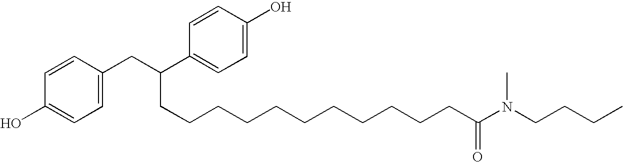 |
| Compd 19 | 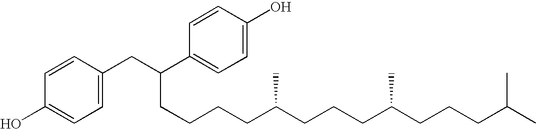 |
| Compd 20 | 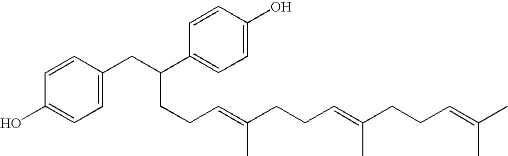 |

In one embodiment, the compound is 7α-[(4R,8R)-4,8,12-trimethyltridecyl]estra-1,3,5-trien-3,17β-diol (NS1 in Table 2). In another embodiment, the compounds are not ICI 182,780 or ICI 164,384.

NS1 is a hybrid structure of 17β-estradiol and Vitamin E, both of which are brain permeable and widely used in humans. The presence of two BBB-penetratable moieties, the estrogenic “head moiety” and the vitamin-like “tail moiety” are likely to increase the potential that the compound will cross the blood brain barrier. In addition, replacement of the “tail moiety” in ICI 182,780, a structural analog of NS1, with a Vitamin E-like hydrophobic chain increases the overall lipid solubility of NS1. While the lipophilicity of NS1 falls out of the range defined by the “Lipinski rule of five” for brain penetration, vitamin E has a similar high lipophilicity, and yet readily enters the brain.

In view of the complexity of the biological features of BBB and the multiple factors that contribute to BBB penetration, by deliberately mimicking the physicochemical properties of vitamin E that may jointly impact its brain entry, including lipophilicity, molecular shape and associated conformational flexibility, and specifically, distribution of a hydrophilic (“head”)/hydrophobic (“tail”) structural balance that may impact the interaction with the BBB membrane-water complex, as revealed by recent membrane-interaction quantitative structure-activity relationship (MI-QSAR) models, NS1 is anticipated to have a similar BBB penetrative ability to vitamin E. Moreover, NS1 has a smaller molecular mass of 496 than ICI 182,780 at 552, and, therefore, falls below the suggested threshold of 500 for a brain-permeable molecule. Most importantly, GOLD docking analyses indicated that NS1 binds to human ERα (hERα) in an energy-favorable fashion, similar to ICI 182,780. In addition, hydrogen bond interactions were observed in both compounds between 3, 17β-OH groups, and the residues, glu353 and His524, respectively, along the ligand binding site in hERα. The similar binding modes and ligand-receptor intermolecular interactions exhibited by ICI 182,780 and NS1 suggested that NS1 would exert a tissue-selective modulation of ER that is consistent with ICI 182,780.

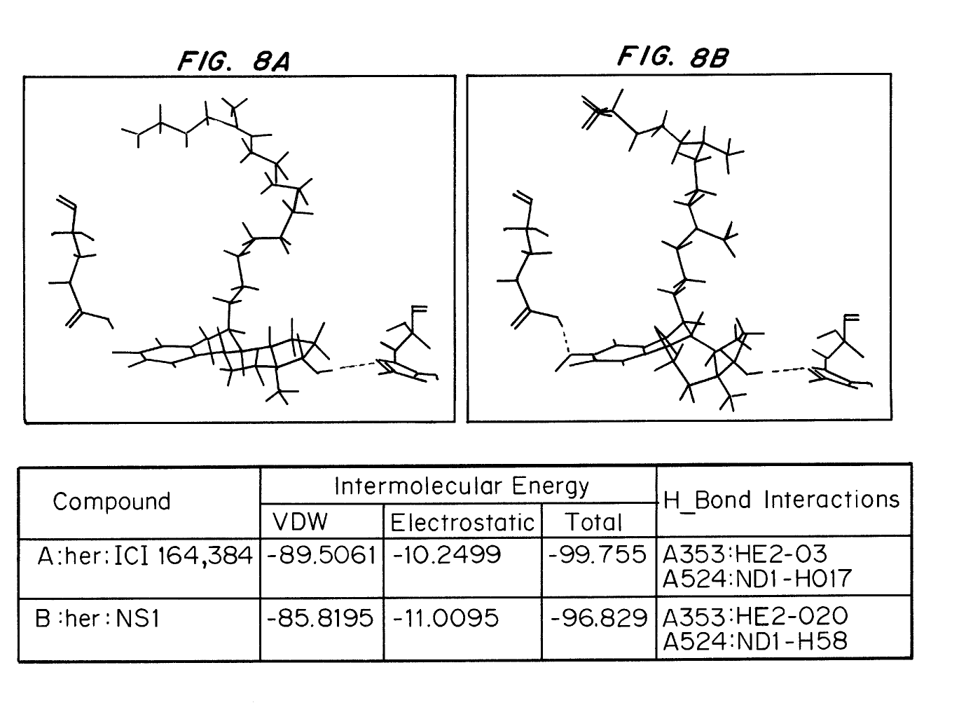

FIGS. 8A and 8B are computer models of the complex structure of human ERα ligand binding domain (LBD) with ICI 164,384 (FIG. 5A) and NS1 (FIG. 8B). Computer modeling of the complex structure of human ERα LBD with ICI 164,384 and NS1 shows NS1 has similar binding mode and orientation as ICI 164,384 in ERα LBD. In addition, NS1 engender strong hydrogen-bond interactions with amino acid residues Glu353 and His 524 as ICI 164,384 does. The modeling is generated by homology modeling based on the crystallographic complex structure of ICI 164,384 with rat ERβ (PDB code: 1HJ1) and by molecular docking with automatic computer docking program GOLD

The compounds can be used in the form of salts derived from inorganic or organic acids. These salts include, but are not limited to, the following: acetate, adipate, alginate, citrate, aspartate, benzoate, benzenesulfonate, bisulfate, butyrate, camphorate, camphorsulfonate, digluconate, cyclopentanepropionate, dodecylsulfate, ethanesulfonate, glucoheptanoate, glycerophosphate, hemi-sulfate, heptanoate, hexamate, fumarate, hydrochloride, hydrobromide, hydroiodide, 2-hydroxyethanesulfonate, lactate, maleate, methanesulfonate, nicotinate, 2-napthalenesulfonate, oxalate, parnoate, pectinate, sulfate, 3-phenylpropionate, picrate, pivalate, propionate, succinate, tartrate, thiocyanate, p-toluenesulfonate and undecanoate. Also, any basic nitrogen-containing groups can be quaternized with agents such as lower alkyl halides, such as methyl, ethyl, propyl, and butyl chloride, bromides, and iodides; dialkyl sulfates like dimethyl, diethyl, dibutyl, and diamyl sulfates, long chain halides such as decyl, lauryl, myristyl and stearyl chlorides, bromides and iodides, aralkyl halides like benzyl and phenethyl bromides, and others. Wafer or oil-soluble or dispersible products are thereby obtained.

Examples of acids which may be employed to form pharmaceutically acceptable acid addition salts include such inorganic acids as hydrochloric acid, sulfuric acid, and phosphoric acid, and organic acids such as oxalic acid, maleic acid, succinic acid and citric acid. Basic addition salts can be prepared in situ during the final isolation and purification of the compounds, or separately by reacting carboxylic acid moieties with a suitable base such as the hydroxide, carbonate or bicarbonate of a pharmaceutically acceptable metal cation or with ammonia, or an organic primary, secondary or tertiary amine. Pharmaceutically acceptable salts include, but are not limited to, cations based on the alkali and alkaline earth metals, such as sodium, lithium, potassium, calcium, magnesium, and aluminum salts, as well as non-toxic ammonium, quaternary ammonium, and mine cations, including, but not limited to ammonium, tetramethylammonium, tetraethylammonium, methylamine, dimethylamine, trimethylamine, triethylamine, ethylamine, and the like. Other representative organic amines useful for the formation of base addition salts include diethylamine, ethylenediamine, ethanolamine, diethanolamine, and piperazine.

The compounds may exist as one or more stereoisomers. As used herein, the term “stereoisomers” refers to compounds made up of the same atoms bonded by the same bonds but having different spatial structures which are not interchangeable. The three-dimensional structures are called configurations. As used herein, the term “enantiomers” refers to two stereoisomers whose molecules are nonsuperimposable mirror images of one another. As used herein, the term “optical isomer” is equivalent to the term “enantiomer”. The terms “racemate”, “racemic mixture” or “racemic modification” refer to a mixture of equal parts of enantiomers. The term “chiral center” refers to a carbon atom to which four different groups are attached. The term “enantiomeric enrichment” as used herein refers to the increase in the amount of one enantiomer as compared to the other. Enantiomeric enrichment is readily determined by one of ordinary skill in the art using standard techniques and procedures, such as gas or high performance liquid chromatography with a chiral column. Choice of the appropriate chiral column, eluent and conditions necessary to effect separation of the enantiomeric pair is well within the knowledge of one of ordinary skill in the art using standard techniques well known in the art, such as those described by J. Jacques, et al., “Enantiomers, Racemates, and Resolutions”, John Wiley and Sons, Inc., 1981. Examples of resolutions include recrystallization of diastereomeric salts/derivatives or preparative chiral chromatography.

B. Assays for Estrogen Receptor Modulating Activity In Vivo and Ex Vivo

The activities of the compounds as estrogen receptor agonists and/or antagonists can be determined using a wide variety of assays known to those having skill in the biochemistry, medicinal chemistry, and endocrinology arts. Several of these assays are discussed below.

Allen-Doisy Test for Estrogenicity

This assay is used to evaluate a test compound for estrogenic activity, and, more specifically, the ability of a test compound to induce an estrogenic cornification of vaginal epithelium (Allen and Doisy 1923; Muhlbock 1940; Terenius 1971). Test compounds are formulated and administered subcutaneously to mature, ovariectomized female rats in test groups. In the third week after bilateral ovariectomy, the rats are primed with a single subcutaneous dose of estradiol to ensure maintenance of sensitivity and greater uniformity of response. In the fourth week, 7 days after priming, the test compounds are administered. The compounds are given in three equal doses over two days (evening of the first day and morning and evening of the second day). Vaginal smears are then prepared twice daily for the following three days. The extent of carnified and nucleated epithelial cells, as well as leucocytes, is evaluated for each of the smears.

Anti-Allen-Doisy Test for Anti-Estrogenicity

This assay is used to evaluate a test compound for anti-estrogenic activity by observation of cornification of the vaginal epithelium of ovariectomized rats after administration of a test compound (Allen and Duisy 1923; Muhlbock 1940; Terenius 1971). Evaluation of anti-estrogenic activity is performed using mature female rats which, two weeks after bilateral ovariectomy, are treated with estradiol to induce a cornification of the vaginal epithelial. This is followed by administration of the test compound in a suitable formulation daily for 10 days. Vaginal smears are prepared daily, starting on the first day of test compound administration and proceeding until one day following the last administration of test compound. The extent of cornified and nucleated epithelial cells and leucocytes is evaluated for each of the smears as above.

Immature Rat Uterotrophic Bioassay for Estrogenicity and Anti-Estrogenicity

Changes in uterine weight in response to estrogenic stimulation can be used to evaluate the estrogenic characteristics of test compounds on uterine tissues (Reel, Lamb et al. 1996; Ashby, Odum et al. 1997). In one example, described below, immature female rats having low endogenous levels of estrogen are dosed with a test compound (subcutaneously) daily for 3 days. The compounds are formulated as appropriate for subcutaneous injection. As a control, 17-β-estradiol is administered alone to one dose group. Vehicle control dose groups are also included in the study. Twenty-four hours after the last treatment, the animals are necropsied, and their uteri excised, nicked, blotted and weighed. Any statistically significant increases in uterine weight in a particular dose group as compared to the vehicle control group demonstrate evidence of estrogenicity.

Estrogen Receptor Antagonist Efficacy in MCF-7 Xenograft Model

This assay is used to evaluate the ability of a compound to antagonize the growth of an estrogen-dependent breast MCF-7 tumor in vivo. Female Ncr-nu mice are implanted subcutaneously with an MCF-7 mammary tumor from an existing in vivo passage. A 17-β-estradiol pellet is implanted on the side opposite the tumor implant on the same day. Treatment with a test compound begins when tumors have reached a certain minimum size (e.g., 75-200 mg). The test compound is administered subcutaneously on a daily basis and the animals are subjected to daily mortality checks. Body weights and tumor volume are determined twice a week starting the first day of treatment. Dosing continues until the tumors reach 1,000 mm³. Mice with tumors larger than 4,000 mg, or with ulcerated tumors, are sacrificed prior to the day of the study determination. The tumor weights of animals in the treatment group are compared to those in the untreated control group as well as those given the estradiol pellet alone.

OVX Rat Model

This model evaluates the ability of a compound to reverse the decrease in bone density and increase in cholesterol levels resulting from ovariectomy. Three-month old female rats are ovariectomized, and test compounds are administered daily by subcutaneous route beginning one day post-surgery. Sham operated animals and ovariectomized animals with vehicle control administered are used as control groups. After 28 days of treatment, the rats are weighed, the overall body weight gains obtained, and the animals euthanized. Characteristics indicative of estrogenic activity, such as blood bone markers (e.g., osteocalcin, bone-specific alkaline phosphatase), total cholesterol, and urine markers (e.g., deoxypyridinaline, creatinine) are measured in addition to uterine weight. Both tibiae and femurs are removed from the test animals for analysis, such as the measurement of bone mineral density. A comparison of the ovariectomized and test vehicle animals to the sham operated and ovariectomized control animals allows a determination of the tissue specific estrogenic/anti-estrogenic effects of the test compounds.

Assays for Estrogen Receptor Modulating Activity In Vitro ERα/ERβ Binding Assays

For evaluation of ERα/ERβ receptor binding affinity, a homogeneous scintillation proximity assay is used. 96-well plates are coated with a solution of either ERα or ERβ. After coating, the plates are washed with PBS. The receptor solution is added to the coated plates, and the plates are incubated. For library screening, [³H]estradiol is combined with the test compounds in the wells of the 96-well plate. Non-specific binding of the radio-ligand is determined by adding estradiol to one of the wells as a competitor. The plates are gently shaken to mix the reagents and a sample from each of the wells is then transferred to the pre-coated ERα or ERβ plates. The plates are sealed and incubated, and the receptor-bound estradiol read directly after incubation using a scintillation counter to determine test compound activity. If estimates of both bound and free ligand are desired, supernatant can be removed and counted separately in a liquid scintillation counter.

ERα/ERβ Transactivation Assays

The estrogenicity of the compounds can be evaluated in an in vitro bioassay using Chinese hamster ovary (“CHO”) cells that have been stably CQ-transfected with the human estrogen receptor (“hER”), the rat oxytocin promoter (“RO”) and the luciferase reporter gene (“LUC). The estrogen transactivation activity (potency ratio) of a test compound to inhibit transactivation of the enzyme luciferase as mediated by the estrogen receptor is compared with a standard and the pure estrogen antagonist.

MCF-7 Cell Proliferation Assays

MCF-7 cells are a common line of breast cancer cells used to determine in vitro estrogen receptor agonist/antagonist activity (Maceregor and Jordan 1998). The effect of a test compound on the proliferation of MCF-7 cells, as measured by the incorporation of 5-bromo-2′-deoxyuridine (t“BrdU”) in a chemiluminescent assay format, can be used to determine the relative agonist/antagonist activity of the test compound. MCF-7 cells (ATCC HTB-22) are maintained in log-phase culture. The cells are plated and incubated in phenol-free medium to avoid external sources of is estrogenic stimulus (MacCregor and Jordan 1998). The test compound is added at varying concentrations to determine an IC₅₀ for the compound. To determine agonist activity, the assay system is kept free of estrogen or estrogen-acting sources. To determine antagonist activity, controlled amounts of estrogen are added.

C. Additional Active Agents

While the compounds can be administered as the sole active pharmaceutical agent, they can also be used in combination with other modulators described herein, and/or in combination with other agents used in the treatment and/or prevention of estrogen receptor-mediated disorders. Alternatively, the compounds can be administered sequentially with one or more such agents to provide sustained therapeutic and prophylactic effects. Suitable agents include, but are not limited to, other SERMs as well as traditional estrogen agonists and antagonists. Representative agents useful in combination with the compounds for the treatment of estrogen receptor-mediated disorders include, for example, tamoxifen, 4-hydroxytamoxifen, raloxifene, toremifene, droloxifene, TAT-59, idoxifene, RU 58,688, EM 139, ICI 164,384, ICI 182,780, clomiphene, MER-25, DES, nafoxidene, CP-336,156, GW5638, LY 139481, LY353581, zuclomiphene, enclomiphene, ethamoxytriphetol, delmadinone acetate, bisphosphonate. Other agents that can be combined with one or more of the compounds include aromatase inhibitors such as, but not limited to, 4-hydroxymdrostenedione, plomestane, exemestane, aminoglutethimide, rogletimide, fadrozole, vorozole, letrozole, and anastrozole.

Still other agents useful in combination with the compounds described herein include, but are not limited to antineoplastic agents, such as alkylating agents. Other classes of antineoplastic agents include antibiotics, hormonal antineoplastics and antimetabolites. Examples of useful alkylating agents include alkyl sulfonates such as busulfan, improsulfan and piposulfan; aziridines, such as a benzodizepa, carboquone, meturedepa and uredepa; ethylenimines and methylmelamines such as altretamine, triethylenemelamine, triethylenephosphoramide, triethylenethiophosphoramide and trimethylolmelamine; nitrogen mustards such as chlorambucil, chlornaphazine, cyclophosphamide, estramustine, iphosphmide, mechlorethamine, mechlorethamine oxide hydrochloride, melphalan, novembichine, phenesterine, prednimustine, trofosfamide, and uracil mustard; nitroso ureas, such as camustine, chlorozotocin, fotemustine, lomustine, nimustine, ranimustine, dacarbazine, mannomustine, mitobronitol, mitolactol and pipobroman.

Additional agents suitable for combination with the compounds include protein synthesis inhibitors such as abrin, aurintricarboxylic acid, chloramphenicol, colicin E3, cycloheximide, diphtheria toxin, edeine A, emetine, erythromycin, ethionine, fluoride, 5-fluorotryptophan, fusidic acid, guanylyl methylene diphosphonate and guanylyl imidodiphosphate, kanamycin, kasugmycin, kirromycin, and O-methyl threonine, modeccin, neomycin, norvaline, pactamycin, paromomycine, puromycin, ricin, alpha-sarcin, shiga toxin, showdomycin, sparsomycin, spectinomycin, streptomycin, tetracycline, thiostrepton and trimethoprim. Inhibitors of DNA synthesis, including alkylating agents such as dimethyl sulfate, mitomycin C, nitrogen and sulfur mustards, MNNG and NMS; intercalating agents such as acridine dyes, actinomycins, adriamycin, anthracenes, benzopyrene, ethidium bromide, propidim diiodide-intertwining, and agents such as distamycin and netropsin, can also be combined with compounds in pharmaceutical compositions. DNA base analogs such as acyclovir, adenine, beta.-1-D-arabinoside, amethopterin, aminopterin, 2-aminopurine, aphidicolin, 8-azaguanine, azaserine, 6-azauracil, 2′-azido-2′-deoxynucleosides, 5-bromodeoxycytidine, cytosine, .beta.-1-D-arabinoside, diazooxynorleucine, dideoxynucleosides, 5-fluorodeoxycytidine, 5-fluorodeoxyuridine, 5-fluorouracil, hydroxyurea and 6-mercaptopurine also can be used in combination therapies with the compounds described herein.

Topoisomerase inhibitors, such as coumermycin, nalidixic acid, novobiocin and oxolinic acid, inhibitors of cell division, including colcemide, colchicine, vinblastine and vincristine; and RNA synthesis inhibitors including actinomycin D, .alpha.-amanitine and other fungal amatoxins, cordycepin (3′-deoxyadenosine), dichlaroribofiaanosyl benzimidazole, rifampicine, streptovaricin and streptolydigin also can be combined with the compounds of the disclosure to provide pharmaceutical compositions. Other agents suitable for combination with the compounds are phytoestrogens, herbal and vitamin sources. A particular example of a vitamin source is methylcobalamin which is a form of vitamin B-12 that is neurologically active. Still more such agents will be known to those having skill in the medicinal chemistry and oncology arts.

In addition, the compounds can be used, either singly or in combination as described above, in combination with other modalities for preventing or treating estrogen receptor-mediated diseases or disorders. Such other treatment modalities include without limitation, surgery, radiation, hormone supplementation, and diet regulation. These can be performed sequentially (e.g., treatment with a compound following surgery or radiation) or in combination (e.g., in addition to a diet regimen).

In another embodiment, the compound is either combined with, or covalently bound to, a cytotoxic agent bound to a targeting agent, such as a monoclonal antibody (e.g., a murine or humanized monoclonal antibody). It will be appreciated that the latter combination may allow the introduction of cytotoxic agents into cancer cells with greater specificity. Thus, the active form of the cytotoxic agent (i.e., the free form) will be present only in cells targeted by the antibody. The compounds may also be combined with monoclonal antibodies that have therapeutic activity against cancer.

The additional active agents may generally be employed in therapeutic amounts as indicated in the PHYSICIANS' DESK REFERENCE (PDR) 53rd Edition (2003), or such therapeutically useful amounts as would be known to one of ordinary skill in the art. The compounds and the other therapeutically active agents can be administered at the recommended maximum clinical dosage or at lower doses. Dosage levels of the active compounds in the compositions may be varied to obtain a desired therapeutic response depending on the route of administration, severity of the disease and the response of the patient. The combination can be administered as separate compositions or as a single dosage form containing both agents. When administered as a combination, the therapeutic agents can be formulated as separate compositions that are given at the same time or different times, or the therapeutic agents can be given as a single composition.

D. Pharmaceutical Compositions

1. Carriers and Excipients

The compounds can be formulated with one or more pharmaceutically acceptable carriers and/or excipients. Formulations are prepared using a pharmaceutically acceptable “carrier” composed of materials that are considered safe and effective and may be administered to an individual without causing undesirable biological side effects or unwanted interactions. The “carrier” is all components present in the pharmaceutical formulation other than the active ingredient or ingredients. The term “carrier” includes but is not limited to diluents, binders, lubricants, disintegrators, fillers, matrix-forming compositions and coating compositions. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio. In one embodiment, the compounds are formulated with one or more carriers or excipients assist the compounds in crossing the blood-brain-barrier. Pharmaceutically acceptable excipients include, but are not limited to, diluents, binders, lubricants, disintegrants, colorants, stabilizers, surfactants, and drug delivery modifiers or enhancers.

Diluents, also referred to as “fillers,” are typically necessary to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powdered sugar.

Binders are used to impart cohesive qualities to a solid dosage formulation, and thus ensure that a tablet or bead or granule remains intact after the formation of the dosage forms. Suitable binder materials include, but are not limited to, starch, pregelatinized starch, gelatin, sugars (including sucrose, glucose, dextrose, lactose and sorbitol), polyethylene glycol, waxes, natural and synthetic gums such as acacia, tragacanth, sodium alginate, cellulose, including hydroxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone.

Lubricants are used to facilitate tablet manufacture. Examples of suitable lubricants include, but are not limited to, magnesium stearate, calcium stearate, stearic acid, glycerol behenate, polyethylene glycol, talc, and mineral oil.

Disintegrants are used to facilitate dosage form disintegration or “breakup” after administration, and generally include, but are not limited to, starch, sodium starch glycolate, sodium carboxymethyl starch, sodium carboxymethylcellulose, hydroxypropyl cellulose, pregelatinized starch, clays, cellulose, alginine, gums or cross linked polymers, such as cross-linked PVP (POLYPLASDONE® XL from GAF Chemical Corp).

Stabilizers are used to inhibit or retard decomposition reactions which include, by way of example, oxidative reactions.

Surfactants may be anionic, cationic, amphoteric or nonionic surface active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surf actants include, but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-b-alanine, sodium N-lauryl-b-iminodipropionate, myristoamphoacetate, lauryl betaine and lauryl sulfobetaine.

If desired, the formulations may also contain minor amount of nontoxic auxiliary substances such as wetting or emulsifying agents, dyes, pH buffering agents, or preservatives.

“Carrier” also includes all components of the coating composition which may include plasticizers, pigments, colorants, stabilizing agents, and glidants. The delayed release dosage formulations may be prepared as described in references such as “Pharmaceutical dosage form tablets”, eds. Liberman et. al. (New York, Marcel Dekker, Inc., 1989), “Remington—The science and practice of pharmacy”, 20th ed., Lippincott Williams & Wilkins, Baltimore, Md., 2000, and “Pharmaceutical dosage forms and drug delivery systems”, 6^{th} Edition, Ansel et. al., (Media, P A: Williams and Wilkins, 1995) which provides information on carriers, materials, equipment and processes for preparing tablets and capsules and delayed release dosage forms of tablets, capsules, and granules.

Examples of suitable coating materials include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, and methacrylic resins that are commercially available under the trade name Eudragit® (Roth Pharma, Westerstadt, Germany), Zein, shellac, and polysaccharides. Additionally, the coating material may contain conventional carriers such as plasticizers, pigments, colorants, glidants, stabilization agents, pore formers and surfactants.

E. Dosage Forms

The compounds are preferably formulated in dosage unit form for ease of administration and uniformity of dosage. The expression “dosage unit form” as used herein refers to a physically discrete unit of conjugate appropriate for the patient to be treated. It will be understood, however, that the total daily usage of the compositions of the present invention will be decided by the attending physician within the scope of sound medical judgment. For any compound, the therapeutically effective dose can be estimated initially either in cell culture assays or in animal models, usually mice, rabbits, dogs, or pigs. The animal model is also used to achieve a desirable concentration range and route of administration. Such information can then be used to determine useful doses and routes for administration in humans. Therapeutic efficacy and toxicity of conjugates can be determined by standard pharmaceutical procedures in cell cultures or experimental animals, e.g., ED50 (the dose is therapeutically effective in 50% of the population) and LD50 (the dose is lethal to 50% of the population). The dose ratio of toxic to therapeutic effects is the therapeutic index and it can be expressed as the ratio, LD50/ED50 Pharmaceutical compositions which exhibit large therapeutic indices are preferred. The data obtained from cell culture assays and animal studies can be used in formulating a range of dosages for human use. The compound can be formulated in a unit dosage form for parenteral, enteral, or topical or transdermal administration.

1. Parenteral Formulations

Injectable preparations, for example, sterile injectable aqueous or oleaginous suspensions may be formulated as known in the art using suitable dispersing or wetting agents and suspending agents. The sterile injectable preparation may also be a sterile injectable solution, suspension, or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils can be employed as a solvent or suspending medium. For this purpose any bland fixed oil can be employed including synthetic mono- or diglycerides. In addition, fatty acids such as oleic acid can be used in the preparation of injectable formulations. The injectable formulations can be sterilized, for example, by filtration through a bacteria-retaining filter, or by incorporating sterilizing agents in the form of sterile solid compositions which can be dissolved or dispersed in sterile water or other sterile injectable medium prior to use.

2. Topical Formulations

Dosage forms for topical or transdermal administration include, but are not limited to, ointments, pastes, creams, lotions, gels, powders, solutions, sprays, inhalants, or patches. The conjugate is admixed under sterile conditions with a pharmaceutically acceptable carrier and any needed preservatives or buffers as may be required. Ophthalmic formulations, ear drops and eye drops can also be prepared. The ointments, pastes, creams and gels may contain, in addition to the conjugates, excipients such as animal and vegetable fats, oils, waxes, paraffins, starch, tragacanth, cellulose derivatives, polyethylene glycols, silicones, bentonites, silicic acid, talc and zinc oxide, or mixtures thereof. Transdermal patches have the added advantage of providing controlled delivery of a compound to the body. Such dosage forms can be made by dissolving or dispensing the conjugates in a proper medium. Absorption enhancers can also be used to increase the flux of the compound across the skin. The rate can be controlled by either providing a rate controlling membrane or by dispersing the conjugates in a polymer matrix or gel.

Powders and sprays can contain, in addition to the conjugates of this, excipients such as lactose, talc, silicic acid, aluminum hydroxide, calcium silicates and polyamide powder, or mixtures of these drugs. Sprays can additionally contain customary propellants such as chlorofluorohydrocarbons.

Compositions for rectal or vaginal administration are preferably suppositories which can be prepared by mixing the conjugate with suitable non-irritating excipients or carriers such as cocoa butter, polyethylene glycol, or a suppository wax which are solid at ambient temperature but liquid at body temperature and therefore melt in the rectum or vaginal cavity and release the conjugate.

3. Enteral Formulations

Enteral formulations include, but are not limited to, oral formulations, mucosal, buccal, sublingual, and pulmonary formulations. The dosage form may be a solid or liquid dosage form.

Solid dosage forms for oral administration include capsules, tablets, pills, powders and granules. In such solid dosage forms, the encapsulated or unencapsulated conjugate is mixed with at least one inert, pharmaceutically acceptable excipient or carrier such as sodium citrate or dicalcium phosphate and/or (a) fillers or extenders such as starches, lactose, sucrose, glucose, mannitol and silicic acid, (b) binders such as, for example, carboxymethyl-cellulose, alginates, gelatin, polyvinylpyrrolidinone, sucrose and acacia, (c) humectants such as glycerol, (d) disintegrating agents such as agar-agar, calcium carbonate, potato or tapioca starch, alginic acid, certain silicates and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay and (i) lubricants such as talc, calcium stearate, magnesium stearate, solid polyethylene glycols, sodium lauryl sulfate and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills and granules can be prepared with coatings and shells such as enteric coatings and other coatings well known in the pharmaceutical formulating art.

Liquid dosage forms for oral administration may include pharmaceutically acceptable emulsions, solutions, suspensions, syrups, and elixirs containing inert diluents commonly used in the art, such as water. Such compositions may also comprise adjuvants, such as wetting agents, emulsifying and suspending agents, cyclodextrins, and sweetening, flavoring, and perfuming agents.

When administered orally, the compounds may be encapsulated in a polymeric or lipid matrix. A variety of suitable encapsulation systems are known in the art (“Microcapsules and Nanoparticles in Medicine and Pharmacy,” Edited by Doubrow, M., CRC Press, Boca Raton, 1992; Mathiowitz and Langer J. Control. Release 5:13, 1987; Mathiowitz et al., Reactive Polymers 6:275, 1987; Mathiowitz et al., J. Appl. Polymer Sci. 35:755, 1988; Langer Acc. Chem. Res. 33:94, 2000; Langer S. Control. Release 62:7, 1999; Uhrich et al., Chem. Rev. 99:3181, 1999; Zhou et al., J. Control. Release 75:27, 2001; and Hanes et al., Pharm. Biotechnol. 6:389, 1995). The compounds may be encapsulated within biodegradable polymeric microspheres or liposomes. Examples of natural and synthetic polymers useful in the preparation of biodegradable microspheres include carbohydrates such as alginate, cellulose, polyhydroxyalkanoates, polyamides, polyphosphazenes, polypropylfumarates, polyethers, polyacetals, polycyanoacrylates, biodegradable polyurethanes, polycarbonates, polyanhydrides, polyhydroxyacids, poly(ortho esters) and other biodegradable polyesters. Examples of lipids useful in liposome production include phosphatidyl compounds, such as phosphatidylglycerol, phosphatidylcholine, phosphatidylserine, phosphatidylethanolamine, sphingolipids, cerebrosides and gangliosides. The encapsulated compounds can be dissolved or dispersed in a pharmaceutically acceptable solvent. Alternatively, the encapsulated compound can be formulated into solid oral dosage forms suitable for oral administration.

The compounds can also be administered in the form of liposomes. As is known in the art, liposomes are generally derived from phospholipids or other lipid substances. Liposomes are formed by mono- or multilamellar hydrated liquid crystals that are dispersed in an aqueous medium. Any non-toxic, physiologically acceptable and metabolizable lipid capable of forming liposomes can be used. The present compositions in liposome form can contain, in addition to a compound, stabilizers, preservatives, excipients. The preferred lipids are the phospholipids and phosphatidyl cholines (lecithins), both natural and synthetic. Methods to form liposomes are known in the art (Prescott 1976).

### III. METHODS OF ADMINISTRATION

The compounds can be administered in a variety of ways including enteral, parenteral, pulmonary, nasal, mucosal and other topical or local routes of administration. For example, suitable modes of administration include oral, subcutaneous, transdermal, transmucosal, iontophoretic, intravenous, intramuscular, intraperitoneal, intranasal, subdural, rectal, vaginal and inhalation.

An effective amount of the compound or composition is administered to treat and/or prevent an estrogen receptor-mediated disorder in a human or animal subject. Modulation of estrogen receptor activity results in a detectable suppression or up-regulation of estrogen receptor activity either as compared to a control or as compared to expected estrogen receptor activity. Effective amounts of the compounds generally include any amount sufficient to detectably modulate estrogen receptor activity by any of the assays described herein, by other activity assays known to those having ordinary skill in the art, or by detecting prevention and/or alleviation of symptoms in a subject afflicted with an estrogen receptor-mediated disorder.

Estrogen receptor-mediated disorders that may be treated include any biological or medical disorder in which estrogen receptor activity is implicated or in which the inhibition of estrogen receptor potentiates or retards signaling through a pathway that is characteristically defective in the disease to be treated. The condition or disorder may either be caused or characterized by abnormal estrogen receptor activity. Representative estrogen receptor-mediated disorders include, for example, osteoporosis, menopause, atherosclerosis, estrogen-mediated cancers (e.g., breast and endometrial cancer), Turner's syndrome, benign prostate hyperplasia (i.e., prostate enlargement), prostate cancer, elevated cholesterol, restenosis, endometriosis, uterine fibroid disease, skin and/or vagina atrophy, Alzheimer's disease and dementia.

Successful treatment of a subject may result in the prevention, inducement of a reduction in, or alleviation of symptoms in a subject afflicted with an estrogen receptor-mediated medical or biological disorder. Thus, for example, treatment can result in a reduction in breast or endometrial tumors and/or various clinical markers associated with such cancers. Treatment of Alzheimer's disease can result in a reduction in rate of disease progression, detected, for example, by measuring a reduction in the rate of increase of dementia.

Alzheimer's disease (ADa), a devastating neurodegenerative condition associated with impaired memory and cognitive function, affects an estimated 4.5 million people in the United States.1 Of those affected with AD, 68% are female and 32% are male. The greater female vulnerability to AD has been associated with the marked decrease in the level of estrogen circulating in postmenopausal women.5,6 In addition to its multifaceted health-promoting effects on a woman's body, such as counteraction of postmenopausal symptoms and preservation of bone density, research over the past two decades has supported the use of estrogen therapy (ET) for the prevention of AD and other age-related neurodegenerative insults when timely initiated based on the “healthy cell bias” of estrogen actions in neurons. However, side effects of the currently available ET, such as neoplasm and thrombogenesis remain serious risks to patients.

The amount of active ingredient that may be combined with the carrier materials to produce a single dosage form will vary depending upon the estrogen-mediated disease, the host treated and the particular mode of administration. It will be understood, however, that the specific dose level for any particular patient will depend upon a variety of factors including the activity of the specific compound employed, the age, body weight, general health, sex, diet, time of administration, route of administration, rate of excretion, drug combination, and the severity of the particular disease undergoing therapy. The prophylactically or therapeutically effective amount for a given situation can be readily determined by routine experimentation and is within the skill and judgment of the ordinary clinician.

For exemplary purposes, a prophylactically or therapeutically effective dose will generally be from about 0.01 mg/kg/day to about 100 mg/kg/day, preferably from about 0.1 mg/kg/day to about 20 mg/kg/day, and most preferably from about 1 mg/kg/day to about 10 mg/kg/day of a estrogen receptor modulating compound, which may be administered in one or multiple doses.

### EXAMPLES

The present invention will be further understood by reference to the following non-limiting examples.

Materials and Methods

The following reagents are prepared using ultra pure water (milli-Q quality):

Culture Medium

Dulbecco's MEM/HAM F 12 powder (12.5 g/l; Gibco, Paisley, UK) is dissolved in water. Sodium bicarbonate (2.5 grams/liter (“g/l”)), L-glutamine (0.36 g/l) and sodium pyruvate (5.5.times.10⁻² g/l) were added. This medium is supplemented with an aqueous mixture (0.50 mil/1 medium) of ethanolamine (2.44 ml/1), sodium selenite (0.9 mg/l), and 2-mercaptoethanol (4.2 ml/l). The pH of the medium is adjusted to 7.0.+.−.0.1 with NaOH or HCl (1 mol/l), and the medium is sterilized by membrane filtration using a filter having 0.2 μm pores. The resulting serum-free culture medium is stored at 4° C.

Antibiotics Solution

Streptomycin sulfate (25 g; Mycofarm, Delft, The Netherlands) and sodium penicillin G (25 g; Mycofarm) are dissolved in 1 l water and sterilized by membrane filtration using a filter having 0.2 micron pores.

Defined Bovine Calf Serum Supplemented (“DBCSS”)

DBCSS (Hyclone, Utah), sterilized by the manufacturer, is inactivated by heating for 30 min at 56° C. with mixing every 5 min. Aliquots of 50 ml and 100 ml are stored at −20° C.

Charcoal-Treated DBCSS (“cDBCSS”)

Charcoal (0.5 g; Norit A) is washed with 20 ml water (3 times) and then suspended in 200 ml Tris buffer. For coating, 0.05 g dextran (T70; Pharmacia, Sweden) is dissolved in a suspension that is stirred continuously for 3 hours at room-temperature. The resulting dextran-coated charcoal suspension is centrifuged for 10 min at 8,000 N/kg. The supernatant is removed and 100 ml DBCSS was added to the residue. The suspension is stirred for 30 min at 45° C. under aseptic conditions. Following stirring, the charcoal is removed by centrifugation for 10 min at 8000 N/kg, The supernatant is sterilized by membrane filtration using a first filter having a pore size of 0.8 μm followed by filtration with a second filter having a pore size of 0.2 μm. The sterilized, heat-inactivated cDBCSS is stored at −20° C.

Tris Buffer

Trimethamine (“Tris”, 1.21 g; 10 mmol) is dissolved in approximately 950 ml water. The solution pH is adjusted to 7.4 using HCl (0.2 mol/l) and the volume is diluted to 1 L with water. This buffer is prepared fresh prior to use.

Luclite Substrate Solution

Luclite luminescence kit, developed for firefly luciferase activity measurements in microtiter plates, is obtained from a commercial source (Packard, Meriden, Conn.). Ten milliliters of the above-described buffer solution is added to each flask of substrate.

Preparation of Transfected Cells

Under aseptic conditions, the above-described culture medium is supplemented with antibiotics solution (2.5 ml/l) and heat-inactivated cDBCSS (50 ml/l) to give complete medium. One vial of the above-described recombinant CHO cells is taken from the seed stack in liquid nitrogen and allowed to thaw in water at approximately 37° C. A Roux flask (80 cc) is inoculated with about 5×10⁵ viable cells/ml in complete medium. The flask is flushed with 5% CO₂ in air until a pH of 7.2-7.4 resulted. The cells are subsequently incubated at 37° C. During this period, the complete medium is refreshed twice.

Following incubation the cell culture is trypsinized and inoculated at 1:10 dilution in a new flask (180 cc cell culturing) and at 5×10³ cells with 100 μl complete medium per well in a 96-well white culture plate for transactivation assays. The 96 well plates are incubated over two days. The cells are grown as a monolayer at the bottoms of the wells and reached confluence after two days. After a cell culture period of 20 passages, new cells are taken from the seed stock in liquid nitrogen.

Animals

The use of animals was approved by the Institutional Animal Care and Use Committee at the University of Southern California (Protocol Number: 10780). Embryonic day 18 Sprague-Dawley rat (Harlan, Indianapolis, Ind.) fetuses were used to obtain primary hippocampal neuronal cultures for in vitro experiments. Young adult (14 to 16-week-old, weighing from 270-290 g) female ovariectomized Sprague-Dawley rats (Harlan) were used for in vivo experiments.

In vitro neuroprotection and associated mechanistic studies were conducted in primary hippocampal neurons obtained from embryonic day 18 rat fetuses. In Vitro Treatments: Test compounds (or combinations) were first dissolved in analytically pure DMSO (10 mM) and diluted in Neurobasal medium to the working concentrations right before treatments.

Statistics

Statistically significant differences between groups were determined by a one-way analysis of variance (ANOVA) followed by a Newman-Keuls post hoc analysis.

### Assays

In Vivo Assays

Immature Rat Uterotrophic Bioassay for Estrogenicity Anti-Estrogenicity

Antiestrogenic activity is determined by the ability of a test compound to suppress the increase in uterine wet weight resulting from the administration of 0.2 μg 17-β-estradiol (“E₂”) per day. Any statistically significant decreases in uterine weight in a particular dose group as compared with the E₂ control group are indicative of anti-estrogenicity.

One hundred forty (140) female pups (19 days old) in the 35-50 g body weight range are selected for the study. On day 19 of age, when the pups weigh approximately 35-50 g, they are body weight-order randomized into treatment pups. Observations far mortality, morbidity, availability of food and water, general appearance and signs of toxicity are made twice daily. Pups not used in the study are euthanized along with the foster dams. Initial body weights are taken just prior to the start of treatment at day 19 of age. The final body weights are taken at necropsy on day 22 of age.

Treatment commences on day 19 of age and continues until day 20 and 21 of age. Each animal is given three subcutaneous (“sc”) injections daily for 3 consecutive days. Three rats in each of the control and mid- to high-level dose test groups are anesthetized with a ketamine/xylazine mixture. Their blood is collected by exsanguination using a 22 gauge needle and 5 ml syringe flushed with 10 USP with sodium heparin/ml through the descending vena cava; and then transferred into a 5 ml green top plasma tube (sodium heparin (freeze-dried), 72 USP units). Plasma samples are collected by centrifugation, frozen at −70° C., and analyzed using mass spectrographic to determine the presence and amount of test compound in the serum. Blood chemistry is also analyzed to determine other blood parameters. The uteri from the rats are excised and weighed. The remaining rats are sacrificed by asphyxiation under CO₂. The uteri from these rats are excised, nicked, blotted to remove fluid, and weighed to the nearest 0.1 mg.

In order to determine whether the test compound significantly affected final body weight, a parametric one-way analysis of variance (ANOVA) is performed (SIGMASTAT version 2.0, available commercially from Jandel Scientific, San Rafael, Calif.). Estrogen agonist and antagonist activity is assessed by comparing uterine wet weights across treatment groups using a parametric ANOVA on loglo transformed data. The data are transformed to meet assumptions of normality and homogeneity of variance of the parametric AWQVA. The F value is determined and a Student-Newman-Kuels multiple range test is performed to determine the presence of significant differences among the treatment groups. The test compound is determined to act as a mixed estrogen agonist/antagonist if the test compound does not completely inhibit the 17-β-estradiol stimulated uterotrophic response.

Estrogen Receptor Antagonist Efficacy In MCF-7 Xenograft Model

MCF-7 human mammary tumors from existing in vivo passages are implanted subcutaneously into 95 female Ncr-nu mice. A 17-β-estradiol pellet (Innovative Research of America) is implanted on the side opposite the tumor. Both implants are performed on the same day.

Treatment is started when the tumor sizes are between 75 mg and 200 mg. Tumor weight is calculated according to the formula for the volume of an ellipsoid,

l×w²/2

where l and w are the larger and smaller dimensions of the tumor and unit tumor density is assumed. The test compounds are administered BID: q7hx2, with one drug preparation per week. The test compounds are stored at +4° C. between injections. The dose of test compound is determined according to the individual animal's body weight on each day of treatment. Gross body weights are determined twice weekly, staring the first day of treatment. Mortality checks are performed daily. Mice having tumors larger than 4,000 mg, mice having ulcerated tumors, as and moribund mice are sacrificed prior to the day of study termination. The study duration is limited to 60 days from the day of tumor implantation but termination could occur earlier as determined to be necessary. Terminal bleeding of all surviving mice is performed on the last day of the experiment. Statistical analysis is performed on the data gathered, including mortality, gross individual and group average body weights at each weighing, individual tumor weights and median group tumor weight at each measurement, the incidence of partial and complete regressions and tumor-free survivors, and the calculated delay in the growth of the median tumor fur each group.

OVX Rat Model

This model evaluates the ability of a compound to reverse the decrease in bone density and increase in cholesterol levels resulting from ovariectomy (Black, Author et al. 1994; Willsan, Author et al. 1997). Three-month old female rats are ovariectomized (“ovx”), and test compounds are administered daily by subcutaneous route beginning one day post-surgery. Sham operated animals and ovx animals with vehicle control administered are used as control groups. After 28 days of treatment, the rats are weighed, the overall body weight gains obtained and the animals euthanized. Blood bone markers (e.g., osteocalcin and bone-specific alkaline phosphatase), total cholesterol, and urine markers (e.g., deoxypyridinoline and creatinine) are measured. Uterine wet weights are also obtained. Both tibiae and femurs are removed from the test animals for peripheral quantitative computed tomography scanning or other measurement of bone mineral density. Data from the ovx and test vehicle animals are compared to the sham and ovx control animals to determine tissue specific estrogenic/antiestrogenic effects of the test compounds.

Assays to Measure Neurogenesis

Sixty-one male Sprague-Dawley rats (Iffa Credo) between 10 and 111 months of age are maintained undisturbed until the behavioral testing. Four weeks before the start of the experiment, 2-month-old rats (n 10) are added to the experiment. Animals are housed individually in plastic cages under a constant light-dark cycle (light on, 800-2000 h) with ad libitum access to food and water. Temperature (22° C.) and humidity (60%) are kept constant. Animals with a bad general health status or tumors are excluded.

Behavioral Testing

Twenty and 3-month-old rats were tested in a Morris water maze (180 cm diameter, 60 cm high; EIC, Bordeaux, France) filled with water (21° C.) made opaque by addition of milk powder. An escape platform is hidden 2 cm below the surface of the water in a fixed location in one of four quadrants halfway between the wall and the middle of the pool. Before the start of training, animals are habituated to the pool without a platform 1 min/day for 3 days. During training, animals are required to locate the submerged platform by using distal extra maze cues. They are tested for four trials per day (90 s with an intertrial interval of 30 s and beginning from three different starts points that vary randomly each day). If an animal does not find the platform, it is set on it at the end of the trial. The time to reach the platform (latency in seconds) and the length of the swim path (distance in centimeters) are measured with a computerized tracking system (VIDEOTRACK, Viewpoint, Lyon, France). To test the visual acuity and the motor functions of the aged rats, after the last day of training, the hidden platform is replaced by a visible platform located in the opposite quadrant, and the animals are tested for 2 additional days.

BrdUrd Injections.

BrdUrd (Sigma), a thymidine analogue incorporated into genetic material during synthetic DNA phase (S phase) of mitotic division, is injected 3 weeks after the end of the behavioral testing. This protocol is chosen to avoid the confounding influence of behavioral training on neurogenesis. Thus, it has been shown that learning modifies the survival of the newly born cells that were labeled before the task. In contrast, the entire procedure of water maze training does not seem to modify cell proliferation. Furthermore, investigation of the relationships between the number of new cells produced during learning and the performance of the animals has been made and no correlations are found in either young or aged rats. Two different doses of BrdUrd are used. In the first and third experiments, rats receive one daily i.p. injection of 50 mg/kg BrdUrd dissolved in phosphate buffer (0.1 M, pH 8.4) during 5 days. In the second experiment, rats receive one daily injection of 150 mg/kg BrdUrd during 5 days.

BrdUrd and Ki67 Staining.

Rats are perfused transcardiacally with paraformaldehyde 1 day (first and second experiments) or 3 weeks (third experiment) after the last BrdUrd injection. After a 24-h postfixation period, 50-pm frontal sections were cut on a vibratome. Free-floating sections are processed according to a standard immunohistochemical procedure. One in ten sections is treated for Ki67 immunoreactivity by using a mouse anti-KT67 monoclonal antibody (1:100, NovoCastra, Newcastle, U.K.). For BrdUrd labeling, adjacent sections are treated with 2 N HCl (30 min at 37° C.), and then rinsed in borate buffer for 5 min (0.1 M, pH=8.4). They are incubated with a mouse monoclonal anti-BrdUrd antibody (1/200, DAKO). Sections are processed in parallel and immunoreactivities are visualized by the biotin-streptavidin technique (ABC kit, DAKO) by using 3,3′-diaminobenzidine as chromogen.

Stereological Analysis.

The number of X-immunoreactive (IR) cells in the left and right dentate gyrus is estimated by using a modified version of the optical fractionator method on a systematic random sampling of every tenth section along the rostrocaudal axis of the hippocampal formation. On each section, all X-IR cells are counted with a X 100 microscope objective, in the granule and subgranular layers of the dentate gyrus and in the hilus excluding those in the outermost focal plane. Resulting numbers are tallied and multiplied by the inverse of the section-sampling fraction (1/ssf=10). Then, the sections are counterstained and the surface of the granule cell layer is measured by using a SAMBA 2640 system (Alcatel System, TITN Answare, Grenoble, France) and the granule cell layer sectional volume is estimated by using the Cavalieri method; Vref=T×ΣA×1/ssf, where T is the mean thickness of the vibratome section (50 pm) and A is the area of the granule and subgranular cell layers. The number of granule cells, as assessed morphologically by hematoxylin staining, is determined by using the optical fractionator method (STEREO INVESTIGATOR software, Micro-BrightField, Williston, Vt.). For each one-in-ten section, granule cells are counted at X 100, in 15×15 pm frames at evenly spaced x-y intervals of 330×330 μm.

Analysis of Phenotype.

To examine the phenotype of BrdUrd-IR cells, one in ten sections are obtained from the second experiment are incubated with the BrdUrd antibody (1/500, Accurate Scientific, Westbury, N.Y.) which is revealed by using a CY3-labeled anti-rat IgG antibody (1/1,000, Jackson ImmunoResearch). Then sections are incubated with a mouse monoclonal anti-NeuN antibody (1/1,000, Chemicon, Euromedex, Souffelweyersheim, France) and bound anti-NeuN monoclonal antibodies are visualized with an Alexa 488 goat anti-rabbit IgG (1/1,000, Jackson ImmunoResearch). The percentage of BrdUrd-labeled cells that expressed NeuN is determined throughout the dentate gyrus by using a confocal microscope with HeNe and argon lasers Nikon PCM 2000). Confocal analysis is restricted to the top of the section where penetration of NeuN antibodies is reliable and all BrdUrd double-labeled cells are examined. Sections are optically sliced in the Z plane by using a 1-μm interval, and cells are rotated in orthogonal planes to verify dauble labeling.

Statistical Analysis.

Relationships between behavioral scores and the number of BrdUrd-IR cells are evaluated by using the Pearson correlation test. Differences between the two groups of aged rats are analyzed with a Student t test or an ANOVA.

In vitro Assays

ERα Binding Assays

ERα receptor (.about.0.2 mg/ml, Affinity Bioreagents) is diluted to about 2×10³ mg/ml in phosphate-buffered saline (“PBS”) at a pH of 7.4. Fifty microliters of the EPα-PBS solution is then added to each the wells of a flashplate. The plates are sealed and stored in the dark at 4° C. for 16-18 hours. The buffered receptor solution is removed just prior to use, and the plates are washed 3 times with 200 microliters per well of PBS. The washing is typically performed using a slow dispense of reagent into the wells to avoid stripping the receptor from the well surface.

For library screening, 150 microliters of 1 nM ³H-estradiol (New England Nuclear, Boston, Mass.) in 20 mM Tris-HCl, 1 mM EDTA, 10% glycerol, 6 mM monothioglycerol, 5 mM KCl, pH 7.8 is mixed with 50 microliters of the test compound (in same buffer) in a 96 well mictrotiter plate, resulting in a final estradiol concentration of 0.6 nM. In addition, several dilutions of estradiol, centered on the IC₅₀ of 1-2 nM, are also added to individual wells to generate a standard curve. The plates are gently shaken to mix the reagents. A total of 150 microliters from each of the wells is added to the corresponding wells of the pre-coated ERα plates. The plates are sealed and the components in the wells are incubated either at room temperature for 4 hours or at 4° C. overnight. The receptor bound ligand is read directly after incubation using a scintillation counter. The amount of receptor bound ligand is determined directly, i.e., without separation of bound from free ligand. If estimates of both bound and free ligand are required, the supernatant is removed from the wells, liquid scintillant is added, and the wells are counted separately in a liquid scintillation counter.

ERβ Binding Assays

ERβ receptor (.about.0.2 mg/ml, Affinity Bioreagents) is diluted to about 2×.10³ mg/ml in phosphate-buffered saline (“PBS”) at a pH of 7.4. Fifty microliters of the ERβ-PBS solution is then added to each the wells of a flashplate. The plates are sealed and are stored in the dark at 4° C. for 16-18 hours. The buffered receptor solution is removed just prior to use, and the plates are washed 3 times with 200 microliters per well of PBS. The washing is typically performed using a slow dispense of reagent into the wells to avoid stripping the receptor from the well surface.

For library screening, 150 microliters of 1 nM ³H-estradiol (New England Nuclear, Boston, Mass.) in 20 mM Tris-HCl, 1 mM EDTA, 10% glycerol, 6 mM monothioglycerol, 5 mM KCl, pH 7.8 was mixed with 50 microliters of the test compound (in same buffer) in a 96 well microtiter plate, resulting in a final estradiol concentration of 0.6 nM. In addition, several dilutions of estradiol, centered on the IC₅₀ of 1-2 nM, were also added to individual wells to generate a standard curve. The plates are then gently shaken to mix the reagents. A total of 150 microliters from each of the wells is added to the corresponding wells of the pre-coated ERβ plates. The plates are sealed and the components in the wells are incubated at room temperature either for 4 hours or at 4° C. overnight. The receptor bound ligand is read directly after incubation using a scintillation counter. The amount of receptor bound ligand is determined directly, i.e., without separation of bound from free ligand. If estimates of both bound and free ligand are required, the supernatant is removed from the wells, liquid scintillant is added, and the wells are counted separately in a liquid scintillation counter.

ERα/ERβ Transactivation Assays

Construction of Transfected CHO Cells

Transfected CHO cells are derived from CHO KI cells obtained from the American Type Culture Collection (“ATCC”, Rockville, Md.). The transfected cells are modified to contain the following four plasmid vectors: (1) pKCRE with DNA for the human estrogen receptor, (2) pAG-60-neo with DNA for the protein leading to neomycin resistance, (3) pRO-LUC with DNA for the rat oxytocin promoter and for firefly luciferase protein, and (4) pDR₂ with DNA for the protein leading to hygromycine resistance. All transformations with these genetically modified CHO cells are performed under rec-VMT containment according to the guidelines of the COGEM (Commissie Genetische Modificatie). Screening was performed either in the absence of estradiol (estrogenicity) or in the presence of estradiol (anti-estrogenicity).

Assays to Assess Neuronal Function

Neuronal Culture Preparation

Primary cultures of hippocampal neurons were obtained from Embryonic Day 18 (El 8d) rat fetuses. Briefly, after dissected from the brains of the rat fetuses, the hippocampi were treated with 0.02% trypsin in Hank's balanced salt solution (137 mM NaCl, 5.4 mM KCl, 0.4 mM KH₂PO₄, 0.34 mM Na₂HPO₄.7H₂0, 10 mM glucose, and 10 mM HEPES) for 5 min at 37° C. and dissociated by repeated passage through a series of fire-polished constricted Pasteur pipettes. Between 2×10⁴ and 4×10⁴ cells were plated onto poly-D-lysine (10 μg/ml)-coated 22 mm coverslips in covered 35 mm petri dishes for morphological analysis, and 1×10⁵ cells/ml were plated onto poly-D-lysine-coated 24-well, 96-well culture plates or 3-5×10⁵ cells/ml onto 0.1% polyethylenimine-coated 60 mm petri dishes for biochemical analyses. Nerve cells were grown in phenol-red free Neurobasal medium (NBM, Invitrogen Corporation, Carlsbad, Calif.) supplemented with B27, 5 U/ml penicillin, 5 μg/ml streptomycin, 0.5 mM glutamine and 25 μM glutamate at 37° C. in a humidified 10% CO₂ atmosphere at 37° C. for the first 3 days and NBM without glutamate afterwards. Cultures grown in serum-free Neurobasal medium yields approximately 99.5% neurons and 0.5% glial cells.

Intracellular Calcium Imaging

The [Ca²⁺]ᵢ in hippocampal neurons was measured by ratiometric Ca²⁺ imaging with the Ca²⁺-sensitive fluorescent dye fura-2. Prior to imaging, hippocampal neurons were loaded with 2 μM fura-2 acetoxymethyl ester (fura-2 AM, Molecular Probes, Inc., Eugene, Oreg.) for 30-45 min at 37° C. in HEPES-Buffered Solution (HBS), containing (in mM): 100 NaCl, 2.0 KCl, 1.0 CaCl₂, 1.0 MgCl₂, 1.0 NaH₂PO₄, 4.2 NaHCO₃, 12.5 HEPES and 10.0 glucose. Excess fura-2 AM dye was removed by washing with HBS and then the neurons were incubated in HBS for 30 min at 37° C. to equilibrate. The coverslip with fura-2 AM-loaded neurons was removed and attached to the coverslip clamp chamber MS-502S (ALA Scientific Instruments, Westbury, N.Y.) for the Ca²⁺ imaging analysis. Neurons were placed on the stage of an inverted microscope (MT-2, Olympus) equipped with epifluorescence optics (20× Nikon), The perfusion solution is HBS and the perfusion system connected to the perfusion chamber was balanced using two variable speed pumps. Imaging was performed at room temperature. Neurons were perfused at a flow rate of 2 ml/min. Fura-2 was excited by a xenon light source at 340 and 380 nm. The emitted fluorescence was filtered through a 520 nm filter, captured with an intensified CCD camera (COHU) and analyzed with InCyt Im2 software (Intracellular Imaging, Cincinnati, Ohio). The concentration of Ca²⁺ was calculated by comparing the ratio of fluorescence at 340 and 380 nm against a standard curve of known [Ca²⁺].

Neurotrophism Measurements

Morphological Analysis

Primary hippocampal neurons grown on poly-D-lysine-coated coverslips were removed from the culture dish and rapidly mounted into a recording chamber. Videomicroscopic recording of neurons was accomplished using a Dage-MTI camera equipped with a Newvicon tube linked to an Olympus BH-2 microscope and a Panasonic time-lapse video recorder (Model AG-6050). Recordings were made using phase-contrast optics with a 40× objective and a 1.50 multiplier with 100 W tungsten source passed through a green filter. Neuron recordings were conducted following 24 h exposure to compounds. Selection of neurons for analysis was random, and all recording and morphological analyses were conducted blind to the experimental condition. Morphological analysis was achieved using a BioQuant Image Analysis system designed for quantitative analysis of cellular morphological features. Cell size was controlled by selecting an equal number of cells from each coverslip that fell within three size categories: small, medium and large. Cell size was determined by the area of the field encompassed by the length of extensions. If a cell encompassed ¼ of the monitor field, it was categorized as small; ½ the field was medium; cells encompassing the entire monitor field or required multiple fields for analysis were categorized as large. Neurons intermediate to these dimensions were graded by the analyst to the closest size category. Number of neurites was defined as the number of extensions greater than 50 μm long emanating directly from the cell body. Neurite length represents the summation of the length of all neuritis/neuron. Branches were operationally defined as any extension that exceeded 10 nm length and occurred along the shaft of the neurite. Branches that occurred as second- or third-order processes were not included in this measure. Branch length represents the summation of the length of all branches present on an individual neuron. The number of bifurcation points represents the total number of points at which branches extend from the neuritic shafts plus those points at which branches extend from other branches for an entire neuron. Microspikes were defined as processes emanating from either neurites or branches that measured less than 10 μm.

Neuroprotection Measurements

Glutamate Exposure

Primary hippocampal neurons were pretreated with compounds for 48 hr followed by exposure to 100 μM glutamate for 5 min at room temperature in HEPES buffer containing 100 mM NaCl, 2.0 mM KCl, 2.5 mM CaCl₂, 1.0 mM MgSO₄, 1.0 mM NaH₂PO₄, 4.2 mM NaHCO₃, 10.0 mM glucose and 12.5 mM T-LEPES. Immediately following glutamate exposure, cultures were washed once with HEPES buffer and replaced with fresh Neurobasal medium containing the test compounds. Cultures were returned to the culture incubator and allowed to incubate for 24 hr prior to cell viability measurements on the following day.

Measurement of LDH Release

Lactate dehydrogenase (LDH) release from the cytosol of damaged cells into the culture medium after glutamate exposure was measured using a Cytotoxicity Detection Assay (Roche Diagnostics Carp., Indianapolis, Ind.) which determines the LDH activity in the culture medium to enzymaticly convert the lactate and NAD⁺ to pyruvate and NADH. The tetrazolium salt produced in the enzymatic reaction was then reduced to red formazan in the presence of H⁺, thereby allowing a colormetric detection for neuronal membrane integrity.

Primary hippocampal neurons grown in 24-well plates were pretreated with compounds for 48 hr prior to exposure to 100 μM glutamate and an additional incubation with compounds for 24 hr, followed by LDH measurements on the following day. The measurement of LDH release was conducted according to the manufacturer's instructions. Briefly, 80 μl of culture medium from each well was transferred to a 96-well plate and 80 μl of Cytotoxicity Detection Reagent was added to incubate far 30 min followed by the addition of 40 μl of 1N HCl to stop the reaction. Colorimetric absorbance was measured with an EL311SX spectrophotometer at 490 nm (Bio-Tek Instruments, Inc., Winooski, Vt.). The test medium in 24-well plates was aspirated off and the protein concentration was determined using the BCA Protein Assay (Pierce Biotechnology, Inc., Rockford, Ill.). LDH release was normalized to protein level per culture before analysis of the data.

Measurement of ATP Level

Intracellular ATP levels were determined by a luciferin/luciferase-based method with the CellTiter-GIo luminescent cell viability assay (Promega Corp., Madison, Wis.), which uses ATP, a required co-factor of the luciferase reaction, producing oxyluciferin and releasing energy in the form of luminescence that is proportional to the amount of ATP present, which further signals the presence of metabolically active cells.

Primary hippocampal neurons seeded into solid white and clear bottom 96-well plates were pretreated with compounds for 48 hr followed by exposure to 100 μM glutamate for 5 min. Cultures were retuned to fresh medium with compounds and incubated far 24 hr prior to ATP measurement. Briefly, half volume of culture medium (100 μl/well) was aspirated off and the same volume of freshly prepared CellTiter-Glo Reagent was added to make the final volume 200 μl in each well. The resulting contents were mixed by agitating on an orbital shaker for 10 min to induce cell lysis that permitted the release of cellular ATP into the medium. The plates were then allowed to incubate at room temperature for an additional 10 min to stabilize the luminescence signal prior to luminescence detection with a Lmax microplate luminometer (Molecular Devices Corp., Sunnyvale, Calif.).

Assessment of Live/Dead Cells by Dual Staining with Calcein am and Ethidium Homodimer

The combined use of Calcein AM and Ethidium Homodimer-1 (Molecular Probes, Inc., Eugene, Oreg.) provides a two-color fluorescence analysis that allows simultaneous determination of live and dead cells with two probes that measure two recognized parameters of cell viability respectively, intracellular esterase activity and plasma membrane integrity. Calcein AM is a fluorogenic esterase substrate that enters the live cells through permeability and is enzymatically hydrolyzed to form the polyanionic dye calcein, which is well retained within live cells due to the intact plasma membranes and produces an intense uniform green fluorescence at 530 nm. Ethidium homodimer is excluded from live cells and only able to enter cells through compromised plasma membranes and binds to nucleic acid by intercalating between the base pairs, producing a bright red fluorescence at 645 nm. Therefore, Calcein AM and ethidium homodimer serve as two indicators for the identification of live and dead cells respectively.

Primary hippocampal neurons seeded into solid black and clear bottom 96-well plates were pretreated with compounds for 48 hr before exposure to glutamate and then incubated with compounds for an additional 24 hr before assessment of the cell viability and cytotoxicity on the following day. Cultures were rinsed with phosphate-buffered saline (PBS) and incubated with the combined 1 μM calcein AM and 2 μM ethidium homodimer PBS solution at room temperature for 30 min. The fluorescence intensities were measured on a SpectraMax GEMINI EM dual-wavelength-scanning microplate spectrofluometer (Molecular Devices Corp., Sunnyvale, Calif.) using appropriate excitation and emission filter combinations (485/530 nm for calcein AM and 530/645 nm for ethidium homodimer).

For microscopic analyses, primary hippocampal neurons grown on glass coverslips were treated with compounds and glutamate as described above, cultures were then rinsed once with PBS and incubated with the combined 1 μM calcein AM and 2 μM ethidium homodimer PBS solution at room temperature for 30 min. Following incubation, the coverslip was removed from the culture dish using a fine tipped forceps, and mounted into a recording chamber covered with the dye solution. The labeled neuronal cells were viewed under an Axiovert 200M Marianas Digital Microscopy Workstation (Intelligent Imaging Innovations, Inc., Denver, Colo.) using a ×40 objective.

Western Immunoblotting

MAP Kinase Phosphorylation

Whole cell lysate were prepared as following: Hippocampal neurons grown on poly-D-lysine-coated culture dishes were treated with compounds for appropriate periods. Treated neurons were washed with cold PBS once and scraped off the dish in 1 ml PBS. Cells were then centrifuged at 5,000 rpm for 5 min, and the pellets were dissolved in the HPA lysis buffer PBS, 1% Triton, 0.2% SDS and protease and a phosphatase inhibitor cocktail containing 1 μg/d antipain, 1 μg/ml leupeptin, 1 μg/ml pepstatin, 10 μg/ml soybean trypsin inhibitor, 1 mM sodium orthovanadate and 1 mM PMSF) and suspended by passage through a 200 μl pipette tip Following incubation at 4° C. for 30-60 min, the samples were centrifuged at 12,000 rpm for 10 min, and the supernatants were the whole cell protein extracts.

Protein concentration was determined by the bicinchoninic acid (BCA) method. An appropriate volume of 2× sample buffer was added to the protein samples, and samples were boiled at 95° C. for 5 min. Samples (25 μg of protein per well) were loaded on a 10% SDS-PAGE get and resolved by standard electrophoresis at 80 V. Proteins were then transferred electrophoretically to Immobilon-P polyvinylidene difluoride membranes overnight at 32 V at 4° C. Membranes were blocked for 1 hr at room temperature in 10% nonfat dry milk in PBS containing 0.05% Tween 20 (PB S-T), incubated with primary antibodies against phospho-ERK1/2 (pTpY^{185/187}, 1:760).

Biosource International, Camarilla, Calif.) and total ERK (1:2, 500, Santa Cruz Bio Tech, Santa Cruz, Calif.) at temperatures and times specified by the antibody providers. The membranes were then incubated with horseradish peroxidase (HRP)-conjugated secondary antibodies (1:3,000, Vector Laboratories, Burlingame, Calif.), and results were visualized by the TMB peroxidase substrate kit (Vector Laboratories). Relative amounts of protein were quantified by optical density analysis using Un-Scan-It software (Silk Scientific, Orem, Utah). After transfer, gels were stained with Coomassie blue (Bio-Rad Laboratories, Hercules, Calif.) to double-check equal protein loading.

CREB phosphorylation

Nuclear lysates were prepared as following: Briefly, hippocampal neurons grown on poly-D-lysine coated culture dishes were treated with compounds for appropriate periods, washed with cold PBS once and scraped into 1 ml PBS. Cells were then centrifuged at 5,000 rpm for 5 min, and the pellet was dissolved in Cytoplasm Extraction buffer (10 mM HEPES, 1 mM EDTA, 60 mM KCl, 0.075% Igepal and protease and phosphatase inhibitor cocktail) and suspended by passage through a 200 μl pipette tip. After 30-45 min of incubation at 4° C., the samples were centrifuged at 5,000 rpm for 5 min to generate the cytoplasmic extract in the supernatant. The supernatant cytoplasmic extract was removed, and Nuclear Extraction buffer (20 mM Tris HCl, 1.5 mM MgCl₂, 420 mM NaCl, 0.2 mM EDTA, 25% glycerol, 0.5% Igepal and protease and phosphatase inhibitor cocktail) was added to the pellet followed by 5M NaCl to break the nuclear membrane. Following 30-45 min of incubation at 4° C., the samples were centrifuged at 12,000 rpm for 10 min to generate a supernatant containing the nuclear extract.

Protein concentration was determined by the BCA method. An appropriate volume of 2× sample buffer was added to the protein samples, and samples were boiled at 95° C. for 5 min. Samples (25 μg of proteins per well) were loaded on a 10% SDSPAGE gel and resolved by standard electrophoresis at 90V. Proteins were then electrophoretically transferred to Immobilon-P PVDF membranes overnight at 32 V at 4° C. Membranes were blocked for 1 hr at mom temperature in 10% non-fat dried milk in PBS containing 0.05% Tween 20 (PBS-T), incubated with appropriate primary antibodies against phospho-CREB (pSER¹³³, mouse monoclonal, 1:2000; Cell Signaling Technology, Beverly, Mass.), CREB (rabbit polyclonal, 1:1000; Cell Signaling Technology, Beverly, Mass.), spinophilin (rabbit polyclonal, 1:000; Upstate Biotechnology, Lake Placid, N.Y.), actin (mouse monoclonal, 1:1000; Santa Cruz Biotechnology, Inc., Santa Cruz, Calif.) or histone H1 (mouse monoclonal, 1:250; Santa Cruz Biotechnology, Inc., Santa Cruz, Calif.) at temperatures and times specified by the antibody providers. All primary antibodies were dissolved in PBS-T with 1% horse serum (for mouse monoclonal antibody) or goat serum (for rabbit polyclonal). After washing in PBS-T, the membranes were incubated with horseradish peroxidase-conjugated anti-mouse IgG (1:5000; Vector Laboratories, Inc., Burlingame, Calif.) in PBS-T with 1% horse serum or anti-rabbit IgG (1:5000; Vector Laboratories, Inc., Burlingame, Calif.) in PBS-T with 1% goat serum for 1 hr. Immunoreactive bands were visualized by TMI3 detection kit (Vector Laboratories. Inc., Burlingame, Calif.) and quantified using Un-Scan-It gel image software (Silk Scientific, Inc., Orem, Utah). Following transfer, gels were stained with Coomassie blue (Bio-rad Laboratories, Hercules, Calif.) to ensure equal protein loading.

Bcl-2 Expression

Primary hippocampal neurons were pretreated with compounds for 48 hr before the cells were lysed by incubation in ice-cold lysis buffer containing: 0.005% SDS, 0.1% Igepal, 0.2 mM sodium orthovanadate, 0.2 mM phenylmethylsulfonylfluoride and protease inhibitor mixture in PBS at 4° C. for 45 min. Cell lysates were centrifuged at 10,000 rpm at 4° C. for 10 min, and the concentration of protein in the supernatant was determined using the BCA Protein Assay (Pierce Biotechnology, Inc., Rockford, Ill.). 25 μg of total protein were diluted in 15 μl 2×SDS containing sample buffer and the final volume was made 30 μl with water. After denaturation on a hot plate at 95-100° C. for 5 min, 25 μl of the mixture were loaded per lane on 10% SDS-polyacrylamide mini-gels followed by electrophoresis at 90V. The proteins were then electro-transferred to polyvinylidene difluoride membranes (Millipore Corp., Bedford, Mass.) from the gels. Nonspecific binding sites were blocked with 5% nonfat dry milk in PBS containing 0.05% Tween-20 (PBS-Tween). Membranes were incubated with the primary monoclonal antibody against Bcl-2 (Zymed Laboratories, Inc., S, San Francisco, Calif.) diluted 1:250 in PB S-Tween with 1% horse serum (Vector Laboratories, Inc., Burlingame, Calif.) overnight at 4° C., then incubated with the secondary horseradish peroxidase (HRP)-conjugated horse anti-mouse IgG (Vector Laboratories, Inc., Burlingame, Calif.) diluted 1:5,000 in PBS-Tween with 1% horse serum for 2 hr at room temperature, and Bcl-2 proteins were visualized by developing the membranes with TMB substrate for peroxidase (Vector Laboratories, Inc., Burlingame, Calif.). β-Actin (Santa Cruz Biotechnology, Inc., Santa Cruz, Calif.) level was determined to ensure equal protein loading, and high-range Precision Protein Standards (Bio-Rad Laboratories, Hercules, Calif.) was used to determine protein sizes. Relative intensities of bands were quantified by optical density analysis using an image digitizing software Un-Scan-Tt version 5.1 (Silk Scientific, Inc., Orem, Utah).

### Example 1

### In Vitro Characterization of NeuroSERM NS1 in Hippocampal Neurons

ER Binding Assays

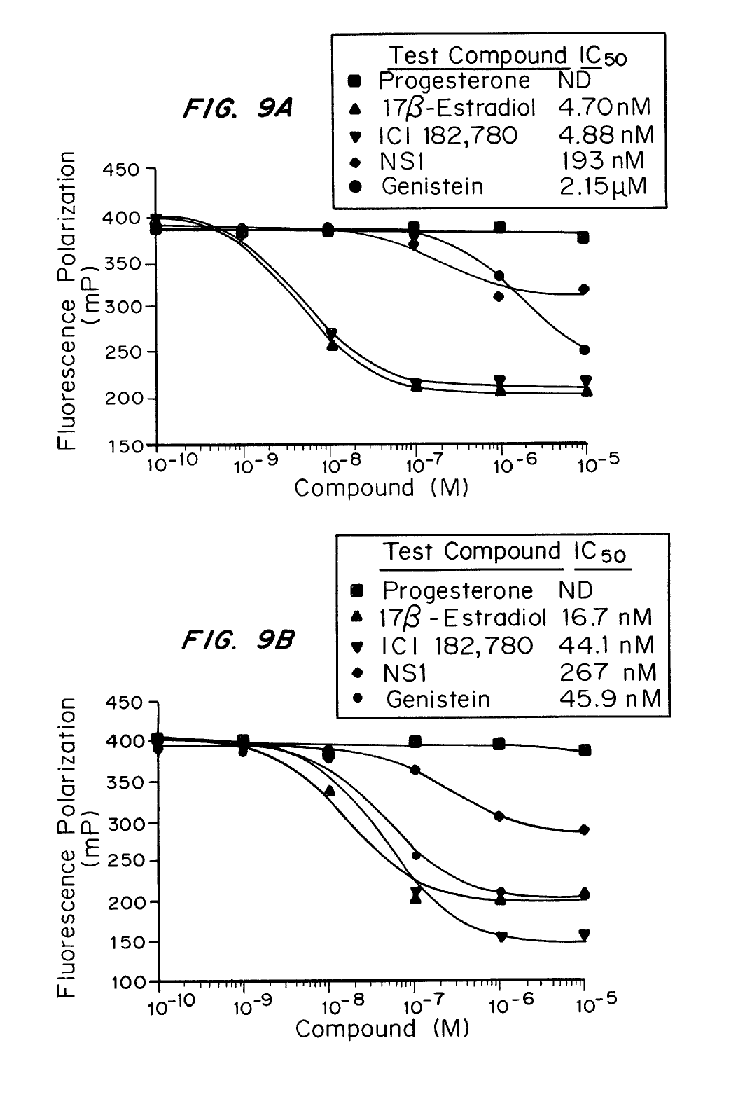

The assays for evaluating the binding efficiency of potential drug candidates with ERα and ERβ are described above. The competition binding curves of NS1 for ERα and ERβ as a function of the concentration of NS1 are shown in FIGS. 9A (Eα) and 9B (Eβ). Progesterone was used as a control and known ER ligands, 17β-estradiol and genistein were used as positive controls. Data were generated with a fluorescence polarization-based competitive binding assay using full-length human ERα and ERβ, and plotted against the logarithm of serially diluted concentrations of the test compounds (or combinations). The ability of the test compounds at serially diluted concentrations (100 pM to 10 iM) to compete with the estrogen ligand EL Red for binding to ERα or ERβ was assessed by a change in polarization values at 535 nm/590 nm excitation/emission.

As expected, the negative control compound, progesterone, does not bind to either ER. The IC50 determined from the binding curves for positive estrogen controls, 17β-estradiol (4.7 and 16.7 nM for ERR and ER\hat{α}, respectively) and ICI 182,780 (4.9 and 44.1 nM for ERα and ERβ, respectively), are consistent with the previously reported values. Moreover, the assay was sufficiently sensitive to differentiate the ERβ-binding preference of the phytoestrogen, genistein, with a 46.8-fold binding selectivity over ERα, which is consistent with results derived from alternative methods such as the radioligand assay. These comparative analyses demonstrate the reliability of this assay in determining the binding profiles of small compounds to both ERs. NS1 comparably bound to both ERα, with a binding IC50 of 193 nM, and ERβ, with a binding IC50 of 267 nM. Although the binding affinity of NS1 to both ERs is approximately 10- to 50-fold lower than those for 17β-estradiol and ICI 182,780, they are well within the therapeutic development range.

Glutamate Exposure

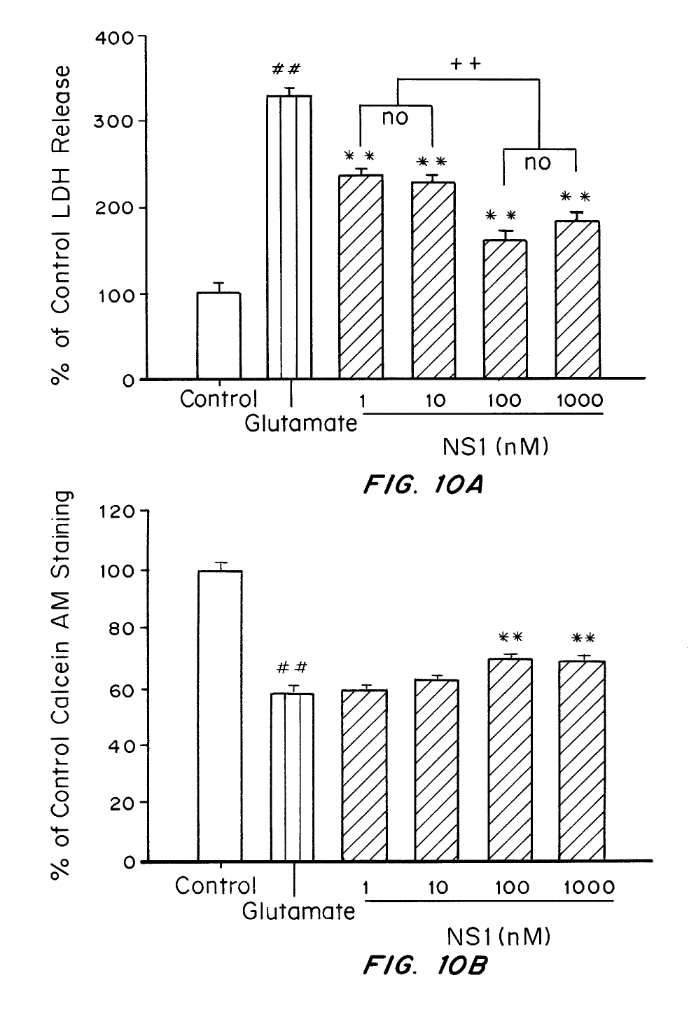

To determine whether 2 would act as an estrogenic agonist in neurons comparable to 17β-estradiol and 1, we first evaluated the activity of NS1 to protect neurons against the neurodegenerative insult, a supraphysiological concentration of glutamate-induced neurotoxicity in cultured rat primary hippocampal neurons. The assay for evaluating neuroprotective efficacy of NS1 after glutamate exposure is described above. The results are shown in FIGS. 10A and 10B.

FIG. 10 shows that NS1 promoted neuronal survival in a concentration-dependent manner. The amount of LDH released in the culture medium induced by 200 iM glutamate was sigmficantly reduced by NS1 at all test concentrations (1-1000 nM), while the efficacy induced by 100-1000 nM was significantly greater than that induced by 1-10 nM of NS1 (++P<0.01). There were no significant differences in LDH release between cultures treated with 1 nM and 10 nM (FIG. 10A, 38.6±3.8% and 44.1±3.8% increase in neuronal membrane integrity compared with glutamate alone-treated cultures, respectively, **P<0.01), and between cultures treated with 100 nM and 1000 nM of NS1 (FIG. 10A, 67.0±4.0% and 59.1±3.7% increase in neuronal membrane integrity compared with glutamate alone-treated cultures, respectively, **P<0.01).

Data shown in FIG. 10B, derived from calcein AM staining of metabolically live neurons in the cultures, revealed a similar trend in neuronal response to serially diluted concentrations of NS1. A significant increase in neuronal viability was observed in cultures treated with 100-1000 nM of NS1 (FIG. 10B, 28.4±3.2% and 25.6±4.4% increase in neuronal metabolic viability compared with glutamate alone-treated cultures, respectively, **P<0.01). In contrast, NS1 at 1-10 nM was insufficient to prevent the loss of neuronal metabolic activity induced by glutamate insult (FIG. 10B, 2.7±3.2% and 9.3±3.7% increase in neuronal metabolic viability compared with glutamate alone-treated cultures, respectively), although 1-10 nM was effective in protecting neurons against glutamate-induced neuronal membrane damage. These differences in outcomes derived from measurements of different biochemical indicators from the same population of neurons suggested that neuronal membrane damage may be more easily protected and repaired than damage to neuronal metabolic function or it may be due to interassay sensitivity. Results of these analyses are consistent with our previous reports for multiple estrogens and ICI 182,780. Moreover, a 10-fold less potency associated with NS1 than 17β-estradiol and ICI 182,780, which exhibited the maximal neuroprotection at 10 nM is consistent with the differences between the ER binding affinity of NS1 and 17β-estradiol and ICI 182,780, suggesting that estrogen-inducible neuroprotective activity is associated with ER-mediated signaling cascades. In summary, these data provided the first line of evidence for an estrogenic agonist profile of NS1 action in neurons consistent with 17β-estradiol and ICI 182,780.

Activation of ERK and AKT

The assay for evaluating activation of ERK and AKT is described above.

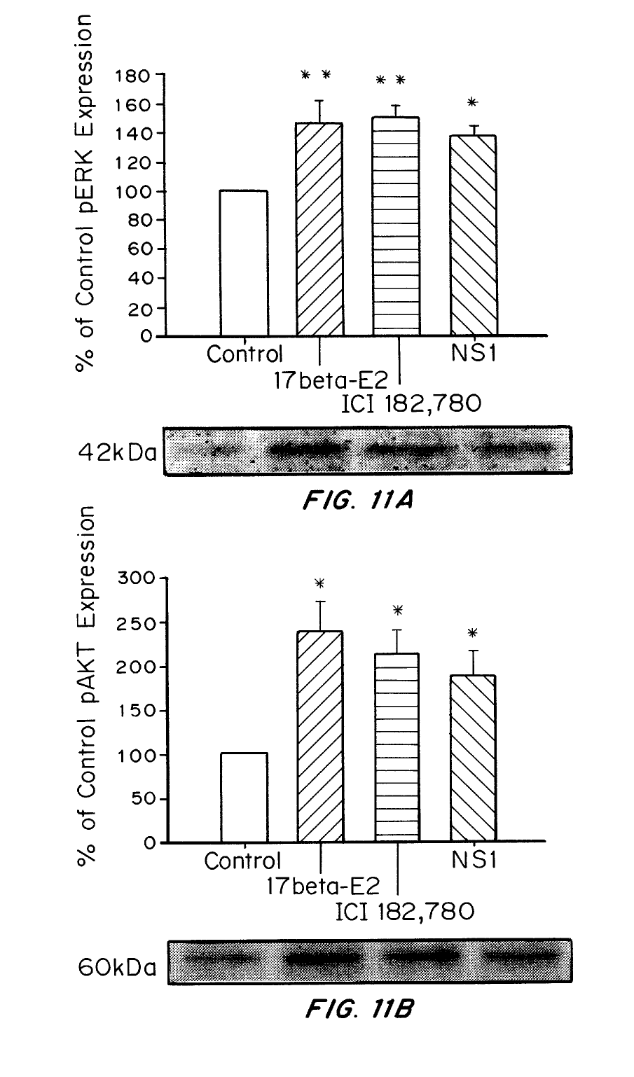

The results for NS1 are shown in FIGS. 11A and 11B. Rat hippocampal neurons grown for 7 DIV were B27 supplement-deprived for 45 min prior to incubation with vehicle alone, 17\hat{α}-estradiol (10 nM), or 2 (100 nM) for 30 min prior to harvesting of proteins for detection of phosphorylated ERK and AKT expression by Western immunoblotting analyses. Total ERK and Akt expression levels in the same protein amples were detected and used as loading controls. Results of these analyses indicated that exposure of neurons to NS1 rapidly induced a significant increase in phosphorylation of both ERK2 and AKT (FIGS. 11A and 11B, 46.3±14.7% and 139.1±33.4% increase compared to vehicle alone treated control cultures, respectively, *P<0.05), with efficacy slightly lower than but not significantly different from that induced by 17β-estradiol (FIGS. 11A and B, 38.0±7.7% and 88.0±27.2% increase compared to vehicle lone-treated control cultures, respectively, *P<0.05).

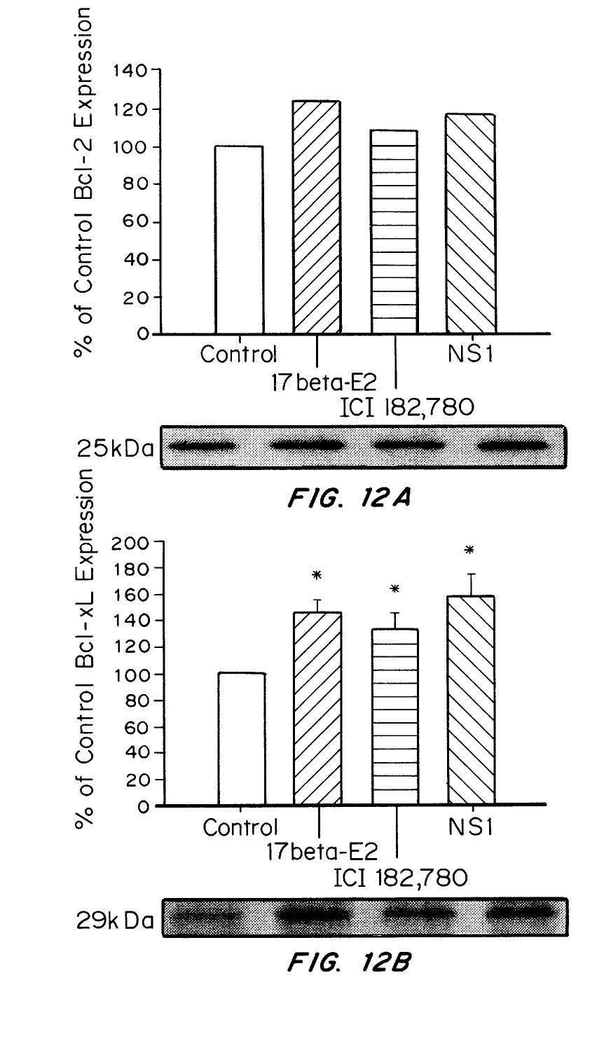

Estrogen upregulation of Bcl-2 family anti-apoptotic proteins Bcl-2 and Bcl-XL has been proposed as one critical component underlying estrogen promotion of neuronal survival. Upregulation of both Bcl-2 and Bcl-XL expression by ICI 182,780 was previously observed as well. Accordingly, we evaluated whether NS1 regulated these proteins in rat primary hippocampal neurons. Neurons grown for 7 DIV were treated with vehicle alone, 17β-estradiol (10 nM) or 2 (100 nM), for 48 h followed by Western immunoblotting analyses. Results of these analyses indicated that NS1 induced a significant increase in both Bcl-2 and Bcl-XL expression in neurons (FIGS. 12A and 12B, 23.4±6.8% and 58.0±22.2% increase compared to vehicle alone treated control cultures, respectively, *P<0.05), with efficacy comparable to 17β-estradiol (FIGS. 12A and 12B, 20.7±1.8% and 46.6±11.6% increase compared to vehicle alone-treated control cultures, respectively, *P<0.05).

In addition to regulating Bcl-2 family anti-apoptotic proteins, estrogen activation of CREB leads to increased expression of spinophilin, a protein that is enriched in the heads of neuronal dendritic spines in hippocampal neurons and which is predictive of estrogen-inducible promotion of neuronal morphogenesis and synaptoplasticity. ICI 182,780 was found to be comparably effective to 17β-estradiol in upregulating spinophilin expression in primary neurons. Based on these earlier findings, we evaluated the impact of NS1 on the expression level of spinophilin, as an indicator of its neurotrophic potential, in comparison with 17β-estradiol. Data indicated that exposure of hippocampal neurons to NS1 (100 nM) for 48 h induced a significant increase in spinophilin expression (61.7±8.2% increase compared to vehicle alone-treated control cultures, *P<0.01). Under the same experimental conditions, 17β-estradiol (10 nM) induced a moderate increase (26.6±6.5% increase compared to vehicle alone-treated control cultures, *P<0.05), which was significantly less than that induced by NS1 (++P<0.01). The present finding is in agreement with earlier studies that showed a similar trend in the difference in magnitude of change in spinophilin expression between 17β-estradiol- and ICI 182,780-treated neurons. These data are promising in that they suggest that NS1 and its structural analogs, a distinct category of ER ligands from the nuclear ER full agonist as represented by 17β-estradiol, have a greater potential in activating mechanisms of neuronal synaptoplasticity and associated memory function. Taken together, results of mechanistic analyses provide the second line of evidence for the estrogenic activity of NS1 in neurons.

### Example 2

### In Vitro Characterization of NeuroSERMs NS2, NS1-1, NS1-2, NS1-3, and NS1-4 in Hippocampal Neurons

Glutamate Exposure

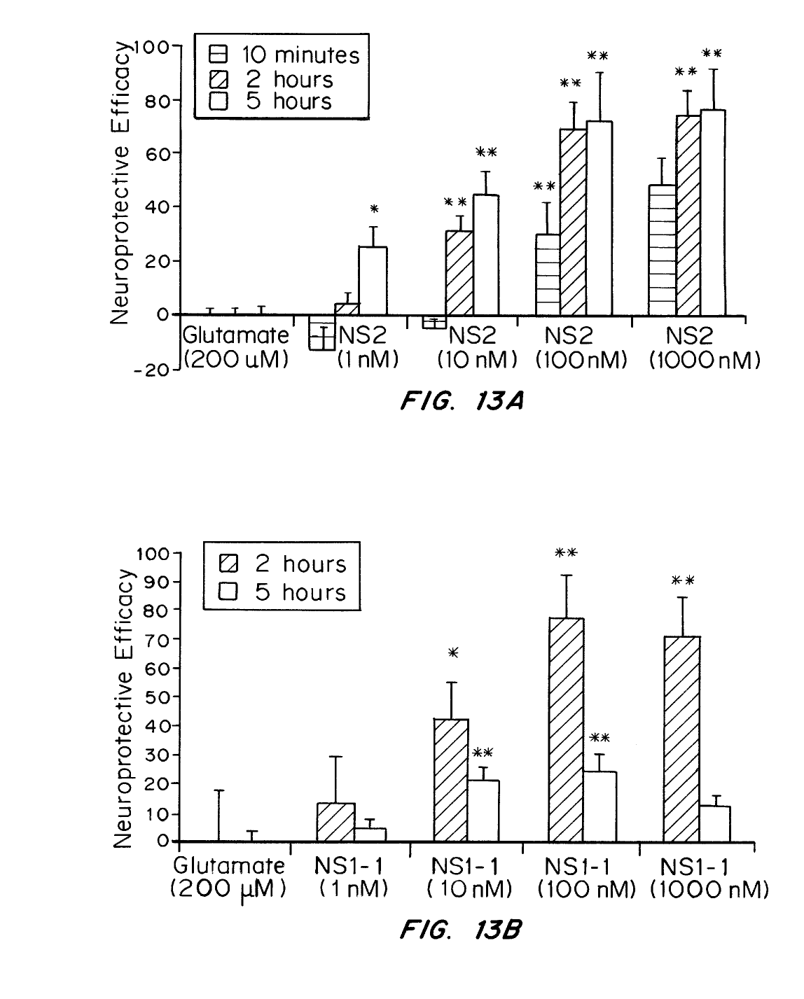

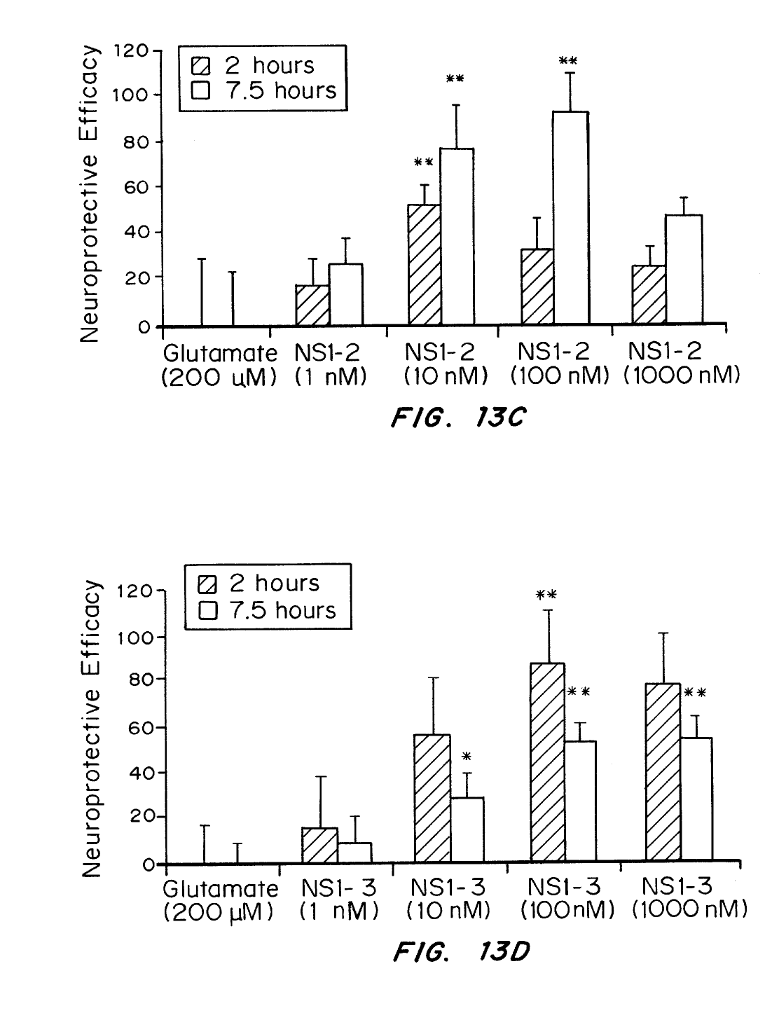

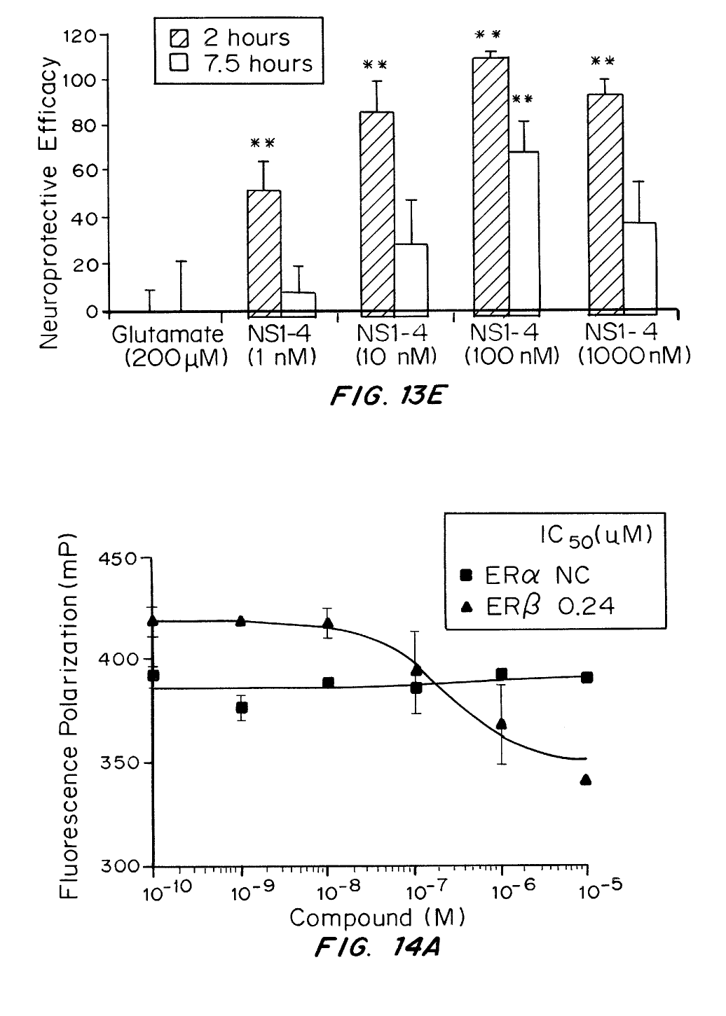

The assay for evaluating neuroprotective efficacy after glutamate exposure is discussed above. The following compounds were evaluated: NS2; NS1-1; NS1-2; NS1-3; and NS1-4. The results are shown in the Tables 4-8 and are presented graphically in FIGS. 13A-E.

Results are presented as neuroprotective efficacy (NE), which is defined as the percentage of neurotoxin-induced toxicity prevented by the test compounds (or combinations) and quantified by the equation:

NE=(Vₜᵣₑₐₜₘₑₙₜ−Vₙₑᵤᵣₒₜₒₓᵢₙ)/(V_{control}−Vₙₑᵤᵣₒₜₒₓᵢₙ)*100%

where Vₜᵣₑₐₜₘₑₙₜ is the individual value from the test compounds (or combinations)-treated cultures, Vₙₑᵤᵣₒₜₒₓᵢₙ is a mean value from glutamate treated cultures, and V_{control} is a mean value from vehicle-treated control cultures.

TABLE 4
Neuroprotective efficacy of NS2

| | 10 minutes | 2 hours after | 5 hours after |
|---|---|---|---|
| | after glutamate | glutamate | glutamate |
| | exposure | exposure | exposure |
| Control | 100 | 100 | 100 |
| Glutamate (200 μM) | 0 | 0 | 0 |
| NS2 (1 nM) | −10.3103 | 3.936783 | 24.93753 |
| NS2 (10 nM) | −3.6302 | 30.95841 | 43.99611 |
| NS2 (100 nM) | 33.68091 | 68.16485 | 71.35349 |
| NS2 (1000 nM) | 40.50652 | 73.84138 | 76.45328 |

TABLE 5
Neuroprotective efficacy of NS1-1

| | 2 hours after | 5 hours after |
|---|---|---|
| | glutamate | glutamate |
| | exposure | exposure |
| Control | 100 | 100 |
| Glutamate (200 μM) | 0 | 0 |
| NS1-1 (1 nM) | 12.7957 | 3.76884 |
| NS1-1 (10 nM) | 41.8183 | 20.8614 |
| NS1-1 (100 nM) | 77.2530 | 23.8415 |
| NS1-1 (1000 nM) | 70.8102 | 11.9900 |

TABLE 6
Neuroprotective efficacy of NS1-2

| | 2 hours after | 7.5 hours after |
|---|---|---|
| | glutamate | glutamate |
| | exposure | exposure |
| Control | 100 | 100 |
| Glutamate (200 μM) | 0 | 0 |
| NS1-2 (1 nM) | 17.5192 | 26.5265 |
| NS1-2 (10 nM) | 51.3940 | 76.6877 |
| NS1-2 (100 nM) | 32.3028 | 93.0520 |
| NS1-2 (1000 nM) | 24.9982 | 47.1665 |

TABLE 7
Neuroprotective efficacy of NS1-3

| | 2 hours after | 7.5 hours after |
|---|---|---|
| | glutamate | glutamate |
| | exposure | exposure |
| Control | 100 | 100 |
| Glutamate (200 μM) | 0 | 0 |
| NS1-3 (1 nM) | 15.44143 | 8.102035 |
| NS1-3 (10 nM) | 55.77281 | 28.06159 |
| NS1-3 (100 nM) | 87.32773 | 51.87228 |
| NS1-3 (1000 nM) | 78.42702 | 53.3893 |

TABLE 8
Neuroprotective efficacy of NS1-4

| | 2 hours after | 7.5 hours after |
|---|---|---|
| | glutamate | glutamate |
| | exposure | exposure |
| Control | 100 | 100 |
| Glutamate (200 μM) | 0 | 0 |
| NS1-4 (1 nM) | 51.85786 | 8.415491 |
| NS1-4 (10 nM) | 86.26971 | 28.79696 |
| NS1-4 (100 nM) | 110.3902 | 69.75865 |
| NS1-4 (1000 nM) | 94.3991 | 38.0418 |

As shown in Tables 4-8 and FIGS. 13A-E, NS2 and NS1-2 show efficacy at early and later time points, whereas the other compounds tend to show efficacy at the early time point. Specifically, NS2 and NS1-2 are neuroprotective at early time points and increases in magnitude at later time points. NS 1-1, NS1-3, and NS1-4 are neuroprotective at early time points with diminished protection at later time points.

ER Binding Assays

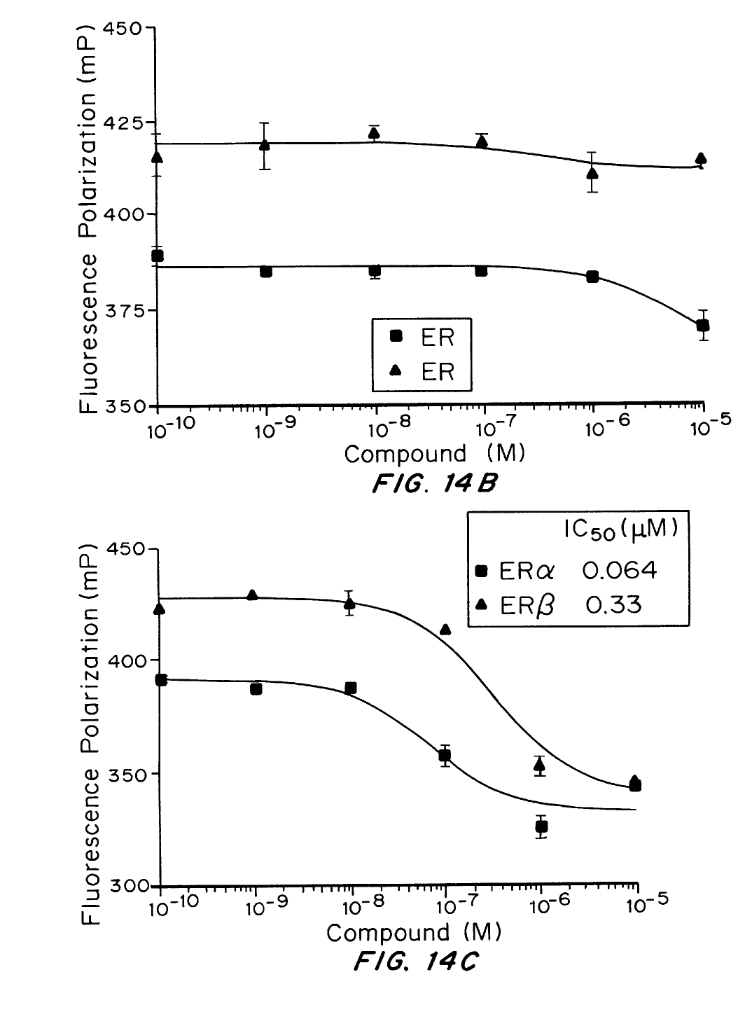

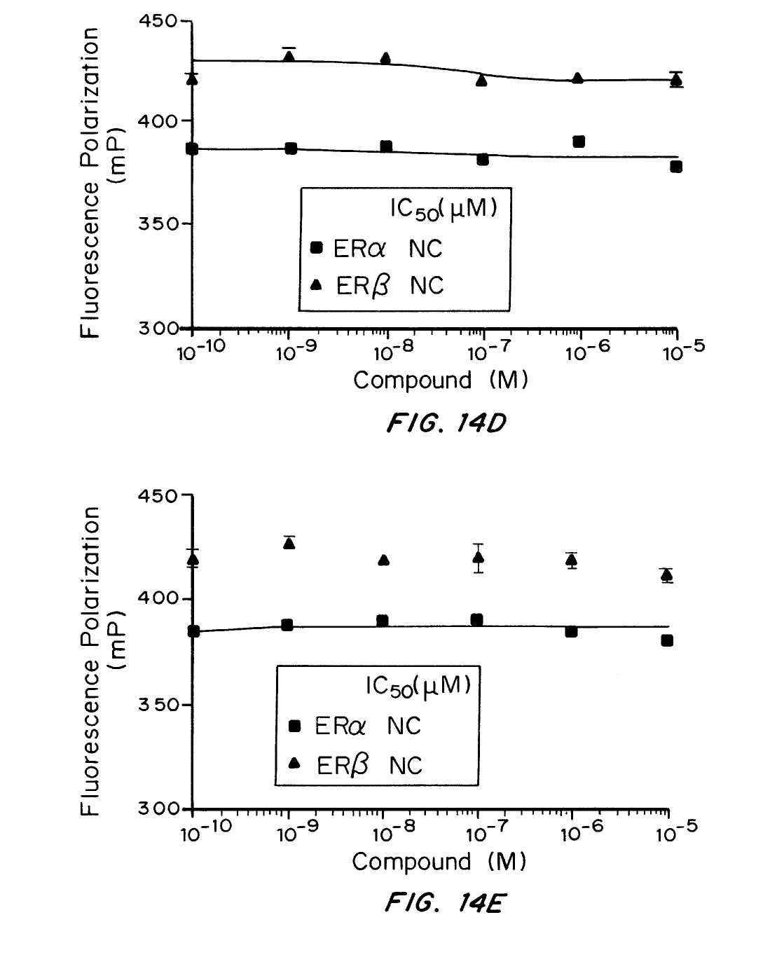

The assays for evaluating the binding efficiency of potential drug candidates with ERα and ERβ are described above. The competition binding curves for ERα, and ERβ for the compounds NS2, NS1-1, NS1-2, NS1-3, and NS1-4 are shown in FIGS. 14A-E. Data were generated with a fluorescence polarization-based competitive binding assay using full-length human ERα and ERb, and plotted against the logarithm of serially diluted concentrations of the test compounds (or combinations).

### Example 3

### Effect of NS1 on MCF-7 Cell Proliferation

MCF-7 cells were seeded onto 24-well culture plates at 1×10⁵/well for 6 hours, followed by incubation with serially diluted concentrations of 17β-estradiol, ICI 182,780 and NS1 for 3 days. Cell proliferation was assessed by MTT measurement at 570 nm. The results are shown in FIGS. 15A-C. Both 17β-estradiol and ICI 182,780 promoted MCF-7 cell proliferation in a concentration dependent manner (FIGS. 15A and 15B). NS1 does not induce MCF-7 cell proliferation (FIG. 15C) and may have an inhibitory effect on proliferation of the breast tumor cells.

Unless defined otherwise, all technical and scientific terms used herein have the same meanings as commonly understood by one of skill in the art to which the disclosed disclosure belongs. Publications cited herein and the materials for which they are cited are specifically incorporated by reference.

Those skilled in the art will recognize, or be able to ascertain using no more than routine experimentation, many equivalents to the specific embodiments described herein. Such equivalents are intended to be encompassed by the following claims

We claim:

1. A compound comprising a head and a tail moiety, wherein the head moiety comprises a steroidal structure, a flavonoid structure, an isoflavonoid structure, a dibenzalkanal, or a 1,4-naphthoquinonyl structure and at least two hydrophilic groups attached approximately at opposite ends of the head moiety, and wherein the tail moiety comprises at least about 10 carbons, wherein the compound crosses the blood brain barrier.

2. The compound of claim 1, wherein at least one of the hydrophilic groups is a hydroxyl group.

3. The compound of claim 1, wherein the at least two hydrophilic groups are hydroxyl groups.

4. The compound of claim 3, wherein the head moiety comprises the steroidal moiety of Formula I and the tail moiety is represented by R4 in Formula 1, 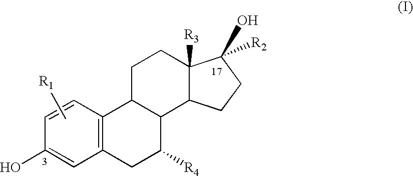
wherein R4 comprises at least 10 carbon atoms and is optionally substituted with one or more chemical groups other than hydrogen, and
wherein R1, R2 and R3 are independently selected from hydrogen, hydroxyl, thio, alkylthio, amino, alkylamino, halo, cyano, lower alkyl, lower alkoxy, lower alkenyl, and lower alkynyl.

5. The compound of claim 4, wherein R1 is a hydrogen atom, a hydroxyl group, a methyl or methoxy group, R2 is a hydrogen atom or an ethynyl group and R3 is a hydrogen atom, a methyl group or a methoxy group.

6. The compound of claim 5, wherein the compound is 7α-[(4R,8R)-4,8,12-trimethyltridecyl]estra-1,3,5-trien-3,17β-diol.

7. The compound of claim 3, wherein the head moiety comprises the flavonoidal moiety of Formula II and the tail moiety is represented by R4, 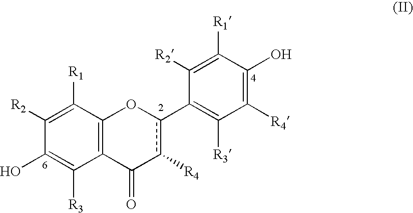
wherein R4 comprises at least 10 carbon atoms and is optionally substituted with one or more chemical groups other than hydrogen,
the bond between carbon 2 and 3 is either a single bond or a double bond; and
wherein R1, R2, R3, R1′, R2′, R3′ and R4′ are independently hydrogen, hydroxyl, thio, alkylthio, amino, alkylamino, halo, cyano, lower alkyl, lower alkoxy, lower alkenyl, and lower alkynyl.

8. The compound of claim 7, wherein the one or more chemical groups are selected from hydroxyl group, methyl group, ethyl group, methoxy group, ethoxyl group, benzoxyl group and halide.

9. The compound of claim 3, wherein the head moiety comprises the isoflavonoidal moiety of Formula III and the tail moiety is represented by R4, 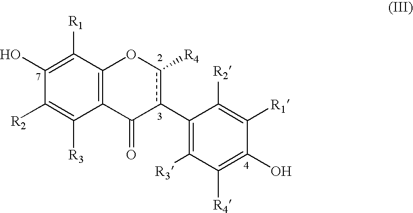
wherein R4 comprises at least 10 carbon atoms and is optionally substituted with one or more chemical groups other than hydrogen,
the bond between carbon 2 and 3 is either a single bond or a double bond; and
wherein R1, R2, R3, R1′, R2′, R3′ and R4′ are independently hydrogen, hydroxyl, thio, alkylthio, amino, alkylamino, halo, cyano, lower alkyl, lower alkoxy, lower alkenyl, and lower alkynyl.

10. The compound of claim 9, wherein the one or more chemical groups are selected from hydroxyl group, methyl group, ethyl group, methoxy group, ethoxyl group, benzoxyl group and halide.

11. The compound of claim 3, wherein the head moiety comprises the dibenzalkane moiety of Formula IV and the tail moiety is represented by R4, 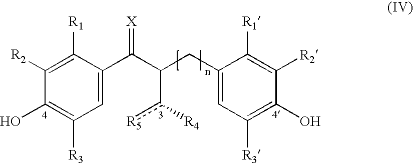
wherein R4 comprises at least 10 carbon atoms and is optionally substituted with one or more chemical groups other than hydrogen,
the bond between carbon 3 and group R5 is either a single bond or double bond; and
wherein R5 is a hydrogen atom, a hydroxyl group, an amino group, a methyl group, a halo group, a thio group or methoxy group if the bond between carbon 3 and group R5 in Formula 4 is a single bond, or R5 is an oxygen atom, a sulphur atom, an oximino group or imino group if the bond between carbon 3 and group R5 is a double bond, and
wherein R1, R2, R3, R1′, R2′ and R3′ are independently hydrogen, hydroxyl, thio, alkylthio, amino, alkylamino, halo, cyano, lower alkyl, lower alkoxy, lower alkenyl, and lower alkynyl,
n is 0, 1 or 2, and
X is O, S, NH or H₂.

12. The compound of claim 11, wherein the one or more chemical groups are selected from hydroxyl group, methyl group, ethyl group, methoxy group, ethoxyl group, benzoxyl group and halide.

13. The compound of claim 3, wherein the head moiety comprises a 1,4-naphthoquinonyl moiety of Formula V and the tail moiety is represented by R4, 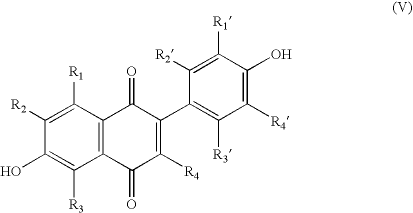
wherein R4 comprises at least 10 carbon atoms and is optionally substituted with one or more chemical groups other than hydrogen, and
wherein R1, R2, R3, R1′, R2′, R3′ and R4′ are independently hydrogen, hydroxyl, thio, alkylthio, amino, alkylamino, halo, cyano, lower alkyl, lower alkoxy, lower alkenyl, and lower alkynyl.

14. The compound of claim 13, wherein the one or more chemical groups is selected from hydroxyl group, methyl group, ethyl group, methoxy group, ethoxyl group, benzoxyl group and halide.

15. The compound of claim 1, wherein the compound is a selective estrogen receptor modulator (SERM) that exhibits agonist effects when bound to the estrogen receptor in brain and exhibits anti-estrogenic effects in breast and uterine, and wherein upon administration of the SERM to a mammal, the SERM is found to be present at least in brain tissue of the mammal.

16. A pharmaceutical composition comprising: a therapeutically effective amount of the compound of claim 1 in combination with a pharmaceutically acceptable carrier.

17. A method for treating an estrogen-related disease or disorder comprising administering to a subject in need thereof an effective amount of the compound of claim 1 in combination with a pharmaceutically acceptable carrier for a period of time effective to treat the estrogen-related disease or disorder.

18. The method of claim 17, wherein the estrogen related disease or disorder is menopause.

19. The method of claim 17, wherein the estrogen related disease or disorder is osteoporosis.

20. The method of claim 17, wherein the estrogen related disease or disorder is breast or endometrial cancers.

21. A method for treating a neurological disease or condition comprising administering to a subject in need thereof an effective amount of the compound of claim 1 in combination with a pharmaceutically acceptable carrier for a period of time effective to treat the neurological disease or condition.

22. The method of claim 21, wherein the neurological disease is Alzheimer's disease.

23. The method of claim 21, wherein the neurological condition results from ischemic injury.

24. The method of claim 21, wherein the neurological disease is vascular dementia.

25. The method of claim 21, wherein the neurological condition is memory loss.
